SEQUENCE LISTING (26 sequences)

SEQ ID NO: 1 - RNA, 20 nt, synthetic construct
  source 1..20: mol_type = other RNA
atgctgctcttaatgatggg

SEQ ID NO: 2 - RNA, 20 nt, synthetic construct
  source 1..20: mol_type = other RNA
gcgtggactatgcctctcgg

SEQ ID NO: 3 - RNA, 20 nt, synthetic construct
  source 1..20: mol_type = other RNA
tcatggataggacccgaatg

SEQ ID NO: 4 - RNA, 20 nt, synthetic construct
  source 1..20: mol_type = other RNA
ggatatgaatcgaagtgcat

SEQ ID NO: 5 - RNA, 77 nt, synthetic construct
  source 1..77: mol_type = other RNA
aacaaagcaccagtggtctagtggtagaatagtaccctgccacggtacagacccgggttcgattcccggctggtgca

SEQ ID NO: 6 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 7 - DNA, 4104 bp, Streptococcus pyogenes
  source 1..4104: mol_type = other DNA
atggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagagccttcgcggctctcatcgctgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgat

SEQ ID NO: 8 - DNA, 963 bp, Petroselinum crispum
  source 1..963: mol_type = other DNA
ggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagc

SEQ ID NO: 9 - DNA, 2614 bp, synthetic construct
  source 1..2614: mol_type = other DNA
ttcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgcatgccaattctagagcggccgcttatcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcaccatgatggtgatgatgatggccctgcagtcccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtacccggggatccacaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctacgtattcttcttcgtattctggcagtataggatcttgtatctgtacattcttcatttttgaacataggttgcatatgtgccgcatattgatctgcttcttgctgagctcacataatacttccatagtttttcccgtaaacattggattcttgatgctacatcttggataattaccttctgcagcccttacattttccaccttacatgtgtcctctatgtctctttcacaatcaccgaccttatcttcttcttttcattgttgtcgtcagtgcttacgtcttcaagattcttttcttcgcctggttcttctttttcaatttctacgtattcttcttcgtattctggcagtataggatcttgtatctgtacattcttcatttttgaacataggttgcatatgtgccgcatattgatctgcttcttgctgagctcacataatacttccatagtttttcccgtaaacattggattcttgatgctacatcttggataattaccttctg

SEQ ID NO: 10 - skipped

SEQ ID NO: 11 - RNA, 18 nt, synthetic construct
  source 1..18: mol_type = other RNA
acaagttcagcgtgtccg

SEQ ID NO: 12 - RNA, 18 nt, synthetic construct
  source 1..18: mol_type = other RNA
tcaccttgatgccgttct

SEQ ID NO: 13 - RNA, 19 nt, synthetic construct
  source 1..19: mol_type = other RNA
gcccctgggaatctgaaag

SEQ ID NO: 14 - RNA, 20 nt, synthetic construct
  source 1..20: mol_type = other RNA
taagttgggtaacgccaggg

SEQ ID NO: 15 - RNA, 20 nt, synthetic construct
  source 1..20: mol_type = other RNA
accgtcagcacagttttgag

SEQ ID NO: 16 - RNA, 20 nt, synthetic construct
  source 1..20: mol_type = other RNA
ccagtcagcaaccttggaag

SEQ ID NO: 17 - RNA, 20 nt, synthetic construct
  source 1..20: mol_type = other RNA
atcgagtatagggcccatcc

SEQ ID NO: 18 - RNA, 20 nt, synthetic construct
  source 1..20: mol_type = other RNA
aggcatagcttcctcccatc

SEQ ID NO: 19 - RNA, 20 nt, synthetic construct
  source 1..20: mol_type = other RNA
agggtagcagttgttggagc

SEQ ID NO: 20 - RNA, 20 nt, synthetic construct
  source 1..20: mol_type = other RNA
aaatcgaccaaccacgacca

SEQ ID NO: 21 - RNA, 20 nt, synthetic construct
  source 1..20: mol_type = other RNA
gacagctggcagagagtgtt

SEQ ID NO: 22 - RNA, 20 nt, synthetic construct
  source 1..20: mol_type = other RNA
gtagcagcagcatttccagc

SEQ ID NO: 23 - RNA, 20 nt, synthetic construct
  source 1..20: mol_type = other RNA
tgggccgattatgaactccg

SEQ ID NO: 24 - RNA, 20 nt, synthetic construct
  source 1..20: mol_type = other RNA
cgcgctctaaatggcttcac

SEQ ID NO: 25 - RNA, 20 nt, synthetic construct
  source 1..20: mol_type = other RNA
tcaattgcgtcctccgtcaa

SEQ ID NO: 26 - RNA, 20 nt, synthetic construct
  source 1..20: mol_type = other RNA
cacaagcaccacccattgtg

(19) United States
(12) Patent Application Publication
(10) Pub. No.: US 2026/0176645 A1
(43) Pub. Date: Jun. 25, 2026

(54) Dual Roles of DA1-EOD1 Module in Regulating Organ Size and Postharvest Quality in Lettuce (Lactuca sativa)

(71) Applicant: UNIVERSITY OF FLORIDA RESEARCH FOUNDATION, INCORPORATED, Gainesville, FL (US)

(72) Inventors: Heqiang HUO, Gainesville, FL (US); Tao JIANG, Gainesville, FL (US)

(21) Appl. No.: 19/196,331

(22) Filed: May 1, 2025

Related U.S. Application Data
(60) Provisional application No. 63/641,247, filed on May 1, 2024.

(51) Int. Cl. C12N 15/82; A01H 5/12

(52) U.S. Cl. CPC C12N 15/8261; A01H 5/12

(57) ABSTRACT

Leaf development, especially in leafy greens, is crucial for crop productivity. In various plants, DA1, a pivotal ubiquitin receptor that negatively regulates leaf growth, and EOD1 (ENHANCER1 OF DA1), an E3 ubiquitin ligases that interacts with DA1 through its RING domain, influence organ size. In this disclosure, in lettuce (Lactuca sativa), homologs genes were identified for both DA1 and EOD1, and da1 single mutants, da1/eod1 double mutants, and DA1 overexpressing lines (DA1ox) were created. The da1/eod1 double mutant exhibited a striking phenotype with larger organ size, accelerated growth, and increased biomass, resulting in higher yields and rapid growth traits pivotal for Controlled Environment Agriculture (CEA). Additionally, the da1/eod1 double mutant displayed a pronounced ‘stay-green’ phenotype, characterized by elevated chlorophyll b levels, which implies extended shelf life, and enhanced postharvest quality, both traits indispensable for leafy vegetables in CEA settings. In contrast, the DA1ox line exhibited early senescence and diminished organ size. Based on these findings that spotlight the nuanced roles of the DA1-EOD1 module in modulating plant growth phases and its potential to revolutionize breeding strategies for improved yield and postharvest longevity in CEA-cultivated leafy greens, this disclosure provides guide RNAs for gene editing of DA1 and EOD1, and transformed lettuce having improved organ size and postharvest quality.

### REFERENCE TO ELECTRONIC SEQUENCE LISTING

The application contains a Sequence Listing which has been submitted electronically in XML format and is hereby incorporated by reference in its entirety. Said .xml file, created on Oct. 10, 2025, is named “10457-584US1_seglist.xml” and is 31036 bytes in size. The sequence listing contained in this .xml file is part of the specification and is hereby incorporated by reference herein in its entirety.

### BACKGROUND

The rising global food demand due to a rapidly growing population is a major challenge for modern sustainable agriculture, and improving crop yield to meet this demand is the key to solving this problem. Lettuce (Lactuca sativa) is a high-value and widely grown leafy vegetable, serving as the major ingredient in ready-to-eat packaged fresh-cut vegetable salads with a market value of $10.78 billion in 2020. As a cool-season crop, lettuce is predominantly produced in California and Arizona in the U.S. This concentrated lettuce production in these two western states requires long-distance transportation to meet the year-round demand nationwide, inevitably leading to high transportation costs and considerable postharvest loss. Moreover, this regional focus on lettuce production makes it vulnerable to various challenges, such as unpredicted disease and pest outbreaks, as well as environmental extremes like floods or severe droughts. Such challenges are further intensified by the global climatic change (Managa et al., 2018; van Delden et al., 2021; Ampim et al., 2022).

Over the past decades, genetic engineering has emerged as a potent tool for enhancing organ size in a multitude of crops, including staples such as rice, wheat, and maize, as well as leafy vegetables like lettuce and spinach (Mao et al., 2010; Mora-Ramirez et al., 2021; Gong et al., 2022; Cai et al., 2021; Li et al., 2013). Therefore, leveraging the existing knowledge to enhance leaf growth and quality through genetic engineering could be a promising strategy to increase lettuce yield and reduce waste, thereby addressing the food insecurity caused by rapid population growth, climatic changes, and diminished resources. Additionally, elucidating the genes and networks that regulate organ size and leaf senescence will provide a foundational basis for genetic enhancements in lettuce.

### SUMMARY

Plant organ size is shaped by cell number and cell size, which are determined by cell proliferation and cell expansion, respectively (Johnson et al., 2011). Early organ growth relies on cell proliferation, while cell differentiation and expansion predominate in the later stage of organ growth (Gonzalez et al., 2012; Andriankaja et al., 2012). Cell proliferation and cell expansion can be modulated by various factors, including transcriptional elements (Zhang et al., 2019), phytohormones (Depuydt et al., 2016) and post-transcriptional modifiers (Dutta et al., 2023). Among them, the DA1-EOD1-mediated ubiquitination pathway plays a pivotal role in regulating protein functions in plants, including modulating growth and developmental processes.

The DA1 (meaning “large” in Chinese) is a ubiquitin-activated endopeptidase that was first identified in Arabidopsis during a genetic screen for mutants with larger seeds. DA1 limits seed and organ size by restricting the duration of cell proliferation (Li et al., 2008). DA1 harbors two ubiquitin interacting motifs (UIM) (Hicke et al., 2005), a LIM domain that is essential for its protease activity, and a conserved C-terminal region that contains the DA1 peptidase motif (FIGS. 1A and C; Dong et al., 2017). Plants with a dominant-negative point mutation in the DA1 gene (da1-1) display enlarged leaves with more cells owing to a prolonged cell proliferation phase (Li et al., 2008; Dong et al., 2017; Vanhaeren et al., 2017). In these mutant plants, a notable increase is not only observed in the sizes of leaves but also in flowers, fruits, and seeds. In contrast, overexpressing DA1 in Arabidopsis resulted in decreased leaf sizes, indicating that DA1 is a negative regulator of leaf growth (Vanhaeren et al., 2017).

In Arabidopsis, seven DA1-related genes (DAR1-7) have been identified, and DAR1 and DAR2 are the ones most closely related to DA1 (Peng et al., 2015). The double mutant of DA1 and DAR1 (da1-ko1_dar1-1) shows a noticeable increase in organ growth similar to da1-1 plants (Li et al., 2008).

The peptidase activity of DA1 is activated by the ENHANCER OF DA1 (EOD1)/BIG BROTHER (BB) and DA2. EOD1/BB and DA2 are RING-type E3 ligases that can interact with DA1 and negatively regulate cell proliferation (Xia et al., 2013; Zhang et al., 2017) (FIG. 1B and D). The RING domain in E3 ligases binds to E2 ubiquitin-conjugating enzymes and facilitates the transfer of ubiquitin to target substrates, thereby promoting degradation of target proteins through the proteasome system (Deshaie et al., 2009). Mutations in EOD1 and DA2 synergistically enhance the da1-1 phenotype, which is characterized by enlarged organs resulting from a prolonged cell proliferation phase (Xia et al., 2013; Dong et al., 2017; Vanhaeren et al., 2017).

DA1-EOD1-mediated ubiquitination pathway regulates organ size by controlling the stability of regulatory proteins. For example, DA1 can modulate the stability of the deubiquitinating enzyme UBIQUITIN SPECIFIC PROTEASE 15 (SOD2/UBP15) (Du et al., 2014; Dong et al., 2017). Notably, overexpression of UBP15 resulted in larger plant organs due to enhanced cell divisions, similar to the da1-1 mutant phenotype. DA1, DAR1, and DAR2 also modulate the stability of transcription factors TEOSINTE BRANCHED 1/CYCLOIDEA/PROLIFERATING CELL NUCLEAR ANTIGEN FACTOR 14 (TCP14) and TCP15 (Dong et al., 2017). These TCPs have been reported to regulate cell proliferation in Arabidopsis. It was observed that loss-of-function of TCP14 and TCP15 in a da1 dar1 dar2 mutant background increases leaf cell size (Peng et al., 2015).

In addition to their role in regulating organ size, DA1 and EOD1 also affect the onset and progression of senescence, as suggested by their role in regulating protein stability via the ubiquitin-proteasome pathway (Li et al., 2008; Dong et al., 2017). Mutants of da1-1 and eod1-2 not only exhibited larger leaves but also showed delayed leaf senescence. These phenotypes were further amplified in the double mutant of da1-1/eod1-2 in Arabidopsis. Conversely, ectopic expression of either DA1 or EOD1 resulted in precocious leaf senescence, indicating their positive role in promoting leaf senescence (Vanhaeren et al., 2017).

In rice, GW2 encodes a RING-type E3 ubiquitin ligase that is homologous to the Arabidopsis DA2, plays a similar role. Dysfunction in rice GW2 caused down-regulated expression of senescence-associated genes (SAGs) and chlorophyll degradation genes, resulting in delayed leaf senescence in dark conditions (Shim et al, 2020). Currently, it remains unclear whether the DA1-EOD1 regulates both developmental progresses, i.e., organ size and senescence, through two divergent pathways or a shared regulatory pathway. In this present disclosure, it was demonstrated that disrupting DA1 and EOD1 genes in lettuce can simultaneously promote leaf growth and improved leaf quality in terms of delay in leaf decay.

Therefore, it is the objective of this disclosure to provide gene editing tools and methods to improve organ size and postharvest quality in lettuce.

In certain embodiments, nucleic acid sequences of guide RNAs that hybridize to the target genes, i.e., lettuce DA1 gene (LsDA1) and lettuce EOD1 gene (LsEOD1), are provided for gene editing, in particular frame shift mutation, by a Cas endonuclease, in particular SpCas9 endonuclease.

In some embodiments, plasmid comprising nucleic acid sequences encoding a gene editing system is provided. The plasmid comprises nucleic acid sequence(s) encoding the guide RNA(s) in addition to a nucleic acid sequence encoding SpCas9 endonuclease as well as nucleic acid sequences encoding NPTII for the selection of Agrobacteria transformed with the plasmid and GFP marker protein for the selection of the plant cells transfected with the plasmid.

In some embodiments, Agrobacterium transformed with the plasmid are provided. In other embodiments, lettuce plant gene-edited using guide RNA targeting DA1 gene (LsDA1) is provided. In other embodiments, lettuce plant gene-edited using guide RNAs targeting DA1 gene (LsDA1) and lettuce EOD1 gene (LsEOD1) are provided.

Further, a method for generating lettuce plants having improved organ size and postharvest quality by using the gene editing system aforementioned is provided.

### BRIEF DESCRIPTION OF THE DRAWINGS

FIG. 1. The DA1 gene family and EOD1 homologs in Arabidopsis and Lettuce. FIG. 1A) Phylogenetic tree and motif of DA1-related genes. FIG. 1B) Phylogenetic tree and motif of EOD1 homologs. FIG. 1C) Protein domains of DA1-related genes. FIG. 1D) Protein domains of RING finger-type E3 ubiquitin ligases EOD1 and DA2. FIG. 1E) The homologs of AtDA1 in lettuce. FIG. 1F) The homologs of AtEOD1 in lettuce.

FIG. 2. Lettuce double mutant da1/eod1 generation. FIG. 2A) The structure of the CRISPR/Cas9 system. FIG. 2B) the location of gRNAs in LsDA1 and FIG. 2C) LsEOD1. FIG. 2D) PCR sanger sequencing results of gene-edited mutations. FIG. 2F) transcriptional analysis of DA1 at two locations of DA1 CDS, with DA1-1 at the C-Terminal, and DA1-2 at the insertion site.

FIG. 3. Leaf size modification in transgenic lettuce mutants. FIG. 3A) Fresh leaf in DA1ox, wide type, da1, and da1/eod1 at 10 WPG. FIG. 3B) Time course of leaf area growth, FIG. 3C) quantitation of the leaf at 8 WPG, and FIG. 3D) fresh weight at 8 WPG of the mutants shown in A). Error bars indicate the mean±SD (n=5). Statistical differences are indicated by different lowercase letters (one-way ANOVA, Tukey's HSD test, P<0.05).

FIG. 4. Overall biomass modification in transgenic lettuce mutants. FIG. 4A) Whole plant size in DA1ox, wide type, da1, and da1/eod1 at 2WPG (juvenile state), and FIG. 4B) 10 WPG (adult stage). FIG. 4C) Quantitation of the aboveground biomass, FIG. 4D) root biomass, and FIG. 4E) time course of plant height of the mutants shown in FIG. 4A). Error bars indicate the mean±SD (n=6). Box plots within violin plots showing the components of descriptive statistics, including medians (white dots), upper and lower quartiles (box edges), minimums and maximums (whiskers), and statistical differences indicated by different lowercase letters (one-way ANOVA, Tukey's HSD test, P<0.05).

FIG. 5. DA1-EOD1 module on lettuce reproductive growth. FIG. 5A) Comparison of the flower phenotypes, and FIG. 5B) seed phenotypes among these genotypes. FIG. 5C), flower length, FIG. 5D) fresh flower weight, FIG. 5E) bolting and flowering time, FIG. 5F) seed area, FIG. 5G) hundred-seed weight, and FIG. 5H) whole seed weight of these genotypes. Box plots within violin plots showing the components of descriptive statistics, including medians (white dots), upper and lower quartiles (box edges), minimums and maximums (whiskers), and statistical differences indicated by different lowercase letters (one-way ANOVA, Tukey's HSD test, P<0.05).

FIG. 6. Cellular morphology of the transgenic and wild-type lettuce. FIG. 6A) Leaf epidermis cells in DA1ox, wide type, da1, and da1/eod1 at 8 WPG. FIG. 6B) Average cell area of the transgenic and the WT lettuce (n=30), and FIG. 6C) cell number per mm2 (n=5) of the mutants shown in A). FIG. 6 D) Relative expression of cell division and expansion marker genes. Box plots within violin plots showing the components of descriptive statistics, including medians (white dots), upper and lower quartiles (box edges), minimums and maximums (whiskers), and statistical differences indicated by different lowercase letters (one-way ANOVA, Tukey's HSD test, P<0.05).

FIG. 7. DA1-EOD1 module mediates leaf senescence and postharvest quality in living plants. FIG. 7A) Dark-induced leaf senescence in DA1ox, wide type, da1, and da1/eod1 at 5 WPG at light and dark conditions for five days. FIG. 7B) Changes in chlorophyll, FIG. 7C) quantitation of the total chlorophyll, FIG. 7D) total carotenoids, FIG. 7E) Fv/Fm ratio, FIG. 7F) Performance Indexes (PI(abs)), and FIG. 7G) electrolyte Leakage (%) after the treatment shown in FIG. 7A). Error bars indicate the mean±SD. Statistical differences are indicated by different lowercase letters (one-way ANOVA, Tukey's HSD test, P<0.05).

FIG. 8. DA1-EOD1 module mediates leaf senescence and postharvest quality in detached leaves. FIG. 8A) Dark-induced leaf senescence in DA1ox, wide type, and da1/eod1 at 5 WPG at room temperature for five days. FIG. 8B) leaf discs are presented as necrotized leaf area, FIG. 8C) Trypan blue staining for the detection of cell death, FIG. 8D) quantitation Cell Death Level. Results are presented as necrotized leaf area compared with the total surface of leaf blades analyzed by ImageJ (1 equals 100% leaf surface), FIG. 8E) leaf biomass with Leaf Area (top), Leaf Fresh Wight per leaf (middle), and Leaf Fresh Wight per cm2 (bottom). Error bars indicate the mean±SD. Statistical differences are indicated by different lowercase letters (one-way ANOVA, Tukey's HSD test, P<0.05). FIG. 8F) DA-related genes involved in leaf senescence. FIG. 8G) The proposed model of DA1-mediated leaf growth and leaf senescence (modified from Zhang et al., 2017).

### DETAILED DESCRIPTION

### A. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The term “lettuce” as used herein refers to garden lettuce (Lactuca sativa), which is a member of the Lactuca (lettuce) genus and the Asteraceae (sunflower or aster) family. The Lactuca (lettuce) genus includes at least 50 species and distributed worldwide. Many species of the genus are common weeds.

As used herein, “explant” refers to a fragment of plant tissue obtained from any part of the plant and used as a starting material to grow a plant in tissue culture. Some examples of explants are cotyledons, epicotyls, fragments or sections of root, stem, shoot, leaf, petals, or apical bud, anthers, or seeds. From there, the explant can be used for regeneration or non-regeneration techniques.

The term “genome editing” or “gene editing” refers to modifying a gene or genes with techniques that employ targeted mutagenesis to activate DNA repair pathways. These techniques include, but are not limited to, those that utilize endonucleases to generate single-strand and double-strand DNA breaks that activate DNA repair pathways. Genome editing techniques may also comprise systems that enable targeted editing at any genomic locus. These targeting systems include, but are not limited to, polypeptides, such as, Transcription Activator-Like Effectors (TALEs) and zinc fingers (ZFs), or nucleic acids, such as, Clustered Regularly Interspaced Short Palindromic Repeats/Cas (CRISPR/CAS) single guide RNAs or NgAgo (Argonaute) single strand DNAs. As used herein, “genome editing” or “gene editing” are interchangeable.

As used herein, the “CRISPR/Cas system” refers to a gene editing system. It has been widely applied to various plant species for trait improvement. CRISPR/Cas system uses two pieces of RNAs, CRISPR RNA (crRNA or spacer) and trans-activating CRISPR RNA (tracrRNA) as well as a Cas endonuclease. The nucleotide sequence of tracrRNA forms hairpin structures for binding to a Cas endonuclease to form an RNP complex (scaffold), and the nucleotide sequence of crRNA spacer directs the RNP complex to a matching target DNA sequence to be edited.

Cas9 is the most well-known Cas DNA endonuclease, a member of Class 2, type II CRISPR/Cas system, which has two active domains, cleaving each of the two DNA strands three nucleotides upstream of the PAM. The five nucleotides upstream of the PAM are defined as the seed region for target recognition. Like other members of the Class 2 CRISPR/Cas systems, including all subtypes of type II and subtype V-B but not subtype V-A or type VI, Cas9 requires a small RNA molecule, i.e., tracrRNA. The tracrRNA is required for the maturation of the pre-crRNA. The tracrRNA is partially complementary to and base pairs with a pre-crRNA forming an RNA duplex. This is cleaved by RNase III, an RNA-specific ribonuclease, to form a crRNA/tracrRNA hybrid. This hybrid acts as a guide for the Cas9 endonuclease. Once the tracrRNA and crRNA form a complex, they bind to the Cas9 endonuclease and direct it to the target sequence via the crRNA.

CRISPR/Cas system can be re-engineered into a more manageable two-component system by fusing the crRNA (spacer) and tracrRNA (scaffold) molecules into a single-guide RNA (sgRNA) or guide RNA (gRNA) that, when combined with a Cas endonuclease, it can find and cut the target DNA specified by the crRNA portion of the guide RNA. Thus, one can change the target gene using the Cas endonuclease by simply changing the sequence of crRNA in the gRNA.

In an aspect, non-limiting examples of an endonuclease for site-specific genome editing provided herein can comprise any RNA-guided Cas endonuclease, including Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof.

The term “target gene” as used herein refers to any nucleotide sequence, which is to be edited by nucleotide deletion, addition, or modification, in the genome of a plant cell to produce genetically transformed/modified cells.

The term “targeting” as used herein refers to Watson-Crick base-pairing or hybridization of crRNA to a genes or genes of a plant cell to be modified by the method and constructs disclosed herein, and is definitely not limited to gene regions, i.e., regions which carry the information for transcription of a mRNA region. These base-pairing or hybridization regions can be regulatory regions of a gene to be modified.

The term “nucleotide” as used herein refers to a sub-unit of a nucleic acid (whether DNA or RNA or an analogue thereof) which may include, but is not limited to, a phosphate group, a 5-carbon sugar group and a nitrogen containing base, as well as analogs of such sub-units. Other groups (e.g., protecting groups) can be attached to the sugar group and nitrogen containing base group. It will be appreciated that, as used herein, the terms “nucleotide” and “nucleoside” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”).

The terms “oligonucleotide,” “nucleotide sequence,” and “nucleic acid sequence” as used herein refer to any polyribonucleotide or polydeoxyribonucleotide that may be unmodified RNA or DNA or modified RNA or DNA. Thus, for instance, oligonucleotides as used herein refers to, among others, single and double-stranded DNA, DNA that is a mixture of single and double-stranded regions, single and double-stranded RNA, and RNA that is mixture of single and double-stranded regions, hybrid molecules comprising DNA and RNA that may be single-stranded or, more typically, double-stranded or a mixture of single and double-stranded regions.

The term “vector” as used herein refers to a nucleic acid capable of transporting another nucleic acid to which it has been linked, usually a DNA molecule that is used as a vehicle to carry a particular foreign nucleic acid sequence—usually DNA into a host/recipient cell where it can be replicated and/or expressed. The vector typically includes features to facilitate the manipulation of DNA as well as a genetic marker for their selective recognition. The most common vectors are DNA plasmids, viruses and artificial chromosomes. Certain vectors are capable of autonomous replication in a host cell into which they are introduced. In general, expression vectors of utility in recombinant DNA techniques are often in the form of plasmids. In the present specification, “plasmid” and “vector” may be used interchangeably as the plasmid is the most commonly used form of vector.

The term “plasmid” as used herein refers to a double-stranded, covalently closed, circular DNA that can be isolated from bacterial cells, which exists in its bacterial hosts as extrachromosomal pieces of DNA that vary in size from 1 kb to >200 kb. Most of the plasmids used in molecular cloning have a multiple cloning site (MCS), also called a polylinker, which is a short segment of DNA which contains various restriction sites a standard feature of engineered plasmids for the insertion of the foreign DNA. In addition, the plasmid should have an origin of replication (ori) site—usually bacterial origin where DNA replication is initiated, marker genes—antibiotics resistance gene for selection and/or screening with antibiotics, and promoters—usually viral origin for gene expression. It should be small in size so that it can easily delivered into the host cell. Plasmids do not generally replicate in the host mammalian cells. By performing a process of DNA transfection or transformation, a plasmid which contains a gene of interest is efficiently delivered to the cells of interest. Numerous plasmid vectors are commercially available, and the modification thereof for specific cloning strategies is well known to the skilled person in the field.

As used herein, a “promoter” is defined as a regulatory DNA sequence that is generally located upstream of a gene and mediates the initiation of transcription by directing RNA polymerase to bind to DNA and initiating RNA synthesis. In some embodiments, the plasmid may comprise more than one RNA polymerase II (pol II) promoters and/or RNA polymerase III (pol III) promoters. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process.

The term “Agrobacterium” as used herein refers to a genus of gram-negative bacteria that uses horizontal gene transfer to cause tumors in plants. Agrobacterium tumefaciens is a soil phytopathogen and the most studied species in this genus, which naturally infects plant wound sites and causes crown gall disease via transfer of a portion of the tumor-inducing (Ti) plasmid, the transfer-DNA (T-DNA), from bacterial cells into host plant cells through a bacterial type IV secretion system (T4SS). (Hwang H H, Yu M, Lai E M. Agrobacterium-mediated plant transformation: biology and applications. Arabidopsis Book. 2017 Oct. 20; 15:e0186).

Ti plasmid is an extrachromosomal molecule of DNA found commonly in the plant pathogen, including Agrobacterium tumefaciens and other species of Agrobacterium such as A. rubi, A. vitis and A. rhizogenes. In the Ti-plasmid, T-DNA is flanked by two repeated sequences of 25-bp length, each, Left and Right Border repeats. These sequences are recognized and nicked by the endonucleolytic proteins VirD1 and VirD2, resulting in a single-stranded form of T-DNA. The VirD2 protein remains covalently linked to the single-stranded T-DNA, and guides it to enter the nucleus. The single-stranded T-DNA is subsequently converted to double-stranded DNA in the nucleus, which may transiently stay in the nucleus to transiently express the genes in the T-DNA or is randomly integrated into the plant genome. (Gelvin S B (2021) Plant DNA Repair and Agrobacterium T-DNA Integration. Int J Mol Sci 22 (16). doi:ARTN 845810.3390/ijms22168458)

The term “transformation” or “transform” refers to genetic transformation that is a process that involves the introduction and expression of foreign genes in a host organism. This expression can result from the extrachromosomal, or episomal presence of genes in nuclei that may persist if the introduced DNA has a mechanism for replication. Bacterial transformation is a process of horizontal gene transfer by which some bacteria take up foreign genetic material (naked DNA) from the environment. For example, in some embodiments, Agrobacterium is transformed with plasmids comprising T-DNA fragment, using heat and thaw methods. Plant genetic transformation (PGT) is a process where DNA is introduced into plant cells, tissues, or organs using molecular and cellular biology methods. PGT comprises steps of delivery of the DNA into a single cell and regeneration into entire fertile plants. In some embodiments, explants of lettuce are genetically ‘transformed’ to have a specific gene modified and express altered phenotypes using Agrobacterium ‘transformed’ with plasmids encoding CRISPR/Cas gene editing system.

The term “transgenic plant” as used herein refers to the plant that have been genetically modified by the insertion of a foreign genetic material (gene(s), DNA sequence(s), etc.) into the chromosome of the cell, while a “transformed plant” is the one whose genome has been genetically modified but not necessarily through insertion of a foreign genetic material. A transgenic plant may have an altered genome containing a DNA sequence or gene from a different species, which expresses a protein that is not native to the plant. The protein encoded by the gene will confer a particular trait or characteristic to that plant.

As used herein, the term “variants” refers to nucleic acid or polypeptide sequences having substantial similarity with a sequence disclosed herein. A variant comprises a deletion and/or addition of one or more nucleotides or peptides at one or more internal sites within the native polynucleotide or polypeptide and/or a substitution of one or more nucleotides or peptides at one or more sites in the native polynucleotide or polypeptide. Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biological activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook”, herein incorporated by reference in its entirety. Alternatively, levels of a marker gene such as green fluorescent protein (GFP) or yellow fluorescent protein (YFP) or the like produced under the control of a promoter operably linked to a nucleotide fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90. Variant nucleotide sequences also encompass sequences derived from a mutagenic and recombinogenic procedure such as DNA shuffling. With such a procedure, one or more different nucleotide sequences can be manipulated to create a new nucleotide sequence. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

### B. Overview

Leaf development, especially in leafy greens, is crucial for crop productivity. It is controlled by a complex network of genes that determine cellular differentiation and organ size. Central to this network is the DA1-EOD1 module, with DA1 functioning as a pivotal ubiquitin receptor that negatively regulates leaf growth, while the E3 ubiquitin ligases EOD1 (ENHANCER1 OF DA1) influences organ size via its interaction with DA1 through its RING domain. To investigate the role of the DA1-EOD1 module in lettuce (Lactuca sativa), lettuce homologs were identified for both DA1 and EOD1, and da1 single mutants, da1/eod1 double mutants, and DA1 overexpressing lines (DA1ox) were created. The da1/eod1 double mutant exhibited a striking phenotype with larger organ size, accelerated growth, and increased biomass, resulting in higher yields and rapid growth traits pivotal for Controlled Environment Agriculture (CEA). Additionally, the da1/eod1 double mutant displayed a pronounced ‘stay-green’ phenotype, characterized by elevated chlorophyll b levels, which implies extended shelf life, and enhanced postharvest quality, both traits indispensable for leafy vegetables in CEA settings. In contrast, the DA1ox line exhibited early senescence and diminished organ size.

B.1. DA1-EOD1 Module in Organ Size Regulation: Insights from Lettuce and Beyond

Organ size is a critical growth characteristic for crop productivity. Extensive studies showed that organ size is determined by cell proliferation and/or cell expansion. A landmark study by Li et al. (2008) uncovered that the enlarged organs in Arabidopsis thaliana da1-1 mutant are primarily caused by increased cell proliferation. Further studies revealed that DA1, DAR1, and DAR2 play a crucial role in restricting the duration of cell proliferation in the early stages of organ development. They achieve this by cleaving and destabilizing various transcription factors that are known to enhance cell proliferation, including TCP 14 and TCP 15 (Li et al., 2008; Peng et al., 2015; Dong et al., 2021). The enhanced cell proliferation in da1 and da1/eod1 Arabidopsis mutants was attributed to relieving their inhibition on these transcription factors.

The DA1 family has highly conserved functions in regulating organ size across plant species. In wheat (Triticum aestivum), silencing of TaDA1 through RNAi resulted in an increase in the size and weight of wheat kernels, while overexpression of TaDA1 the opposite effect. They further demonstrated that TaDA1 restricts the proliferation of maternal pericarp cells (Liu et al., 2020). Overexpression of mutated ZmDA1 or mutated ZmDAR1 in maize (Zea mays) increases kernel yield (Xie et al., 2018).

The two RING-type E3 ligases, DA2 and ENHANCER OF DA1 (EOD1)/BIG BROTHER (BB), also control seed and organ size by physically interacting with DA1 (Li et al., 2008; Xia et 5 al., 2013). In Arabidopsis, loss of function mutations in both DA2 and EOD1/BB results in larger seeds and organs, suggesting that DA2 and EOD1/BB act as inhibitors of seed and organ size.

Similarly, mutation in the homolog of DA2 in rice can increase grain yield (Song et al., 2007), whereas heterologous overexpression of rice DA2 in Arabidopsis resulted in smaller seeds and organs (Xia et al., 2013). In soybeans, homozygous soybean mutants of GmEOD1 generated by CRISPR/Cas9 gene editing also exhibited larger seed size and weight (Yu et al., 2023). It is worth noting that either eod1 or da2-1 mutations synergistically enhance the seed and organ size in Arabidopsis da1-1 single mutant, suggesting the interactive role between EOD1, DA2, and DA1 in controlling seed size through modulating similar downstream targets (Xia et al., 2013).

Intriguingly, the DA1 peptidase is activated by DA2 and EOD1/BB. Once activated, DA1 can then cleave diverse growth regulators, including TCP 14 and TCP 15, thereby regulating seed and organ growth. Furthermore, the activated DA1 peptidase can also cleave and destabilize EOD1/BB and DA2, creating a self-regulatory feedback loop (Xia et al., 2013; Dong et al., 2017).

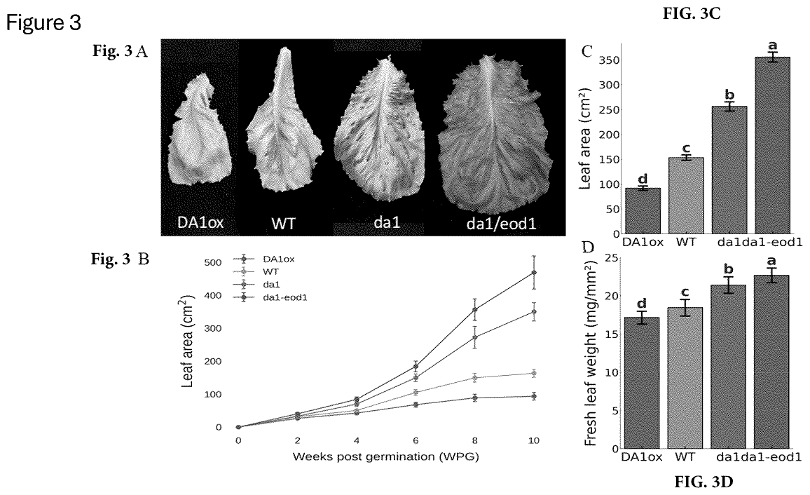

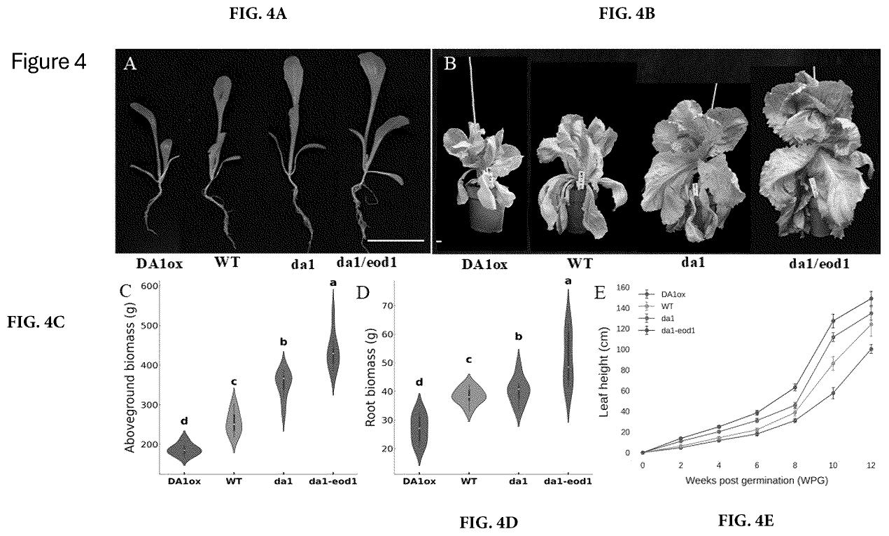

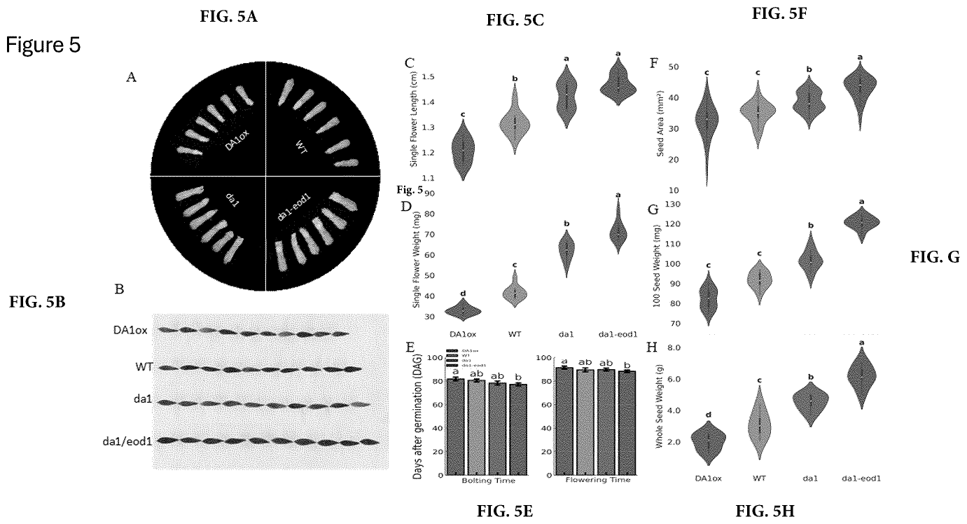

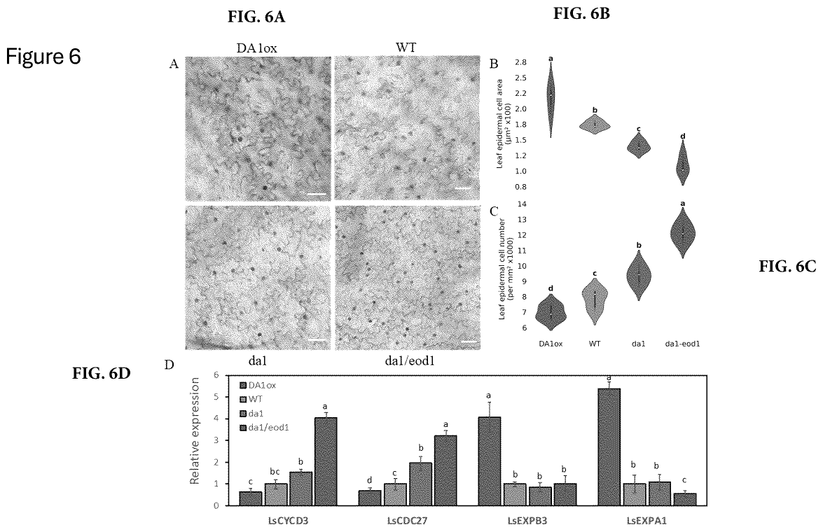

In this disclosure, da1 single mutants and da1/eod1 double mutants were successfully created in lettuce. The results showed that both da1 and da1/eod1 mutants demonstrated significantly larger leaf, flower, and seed sizes compared to the wild type (FIG. 3, FIG. 4, FIG. 6). Similar to the observation in Arabidopsis, the lettuce EOD1 mutation also exhibited an additive effect on the da1 mutant, resulting in notably larger organ sizes than those observed in the da1 single mutant. Additionally, the histological studies have uncovered that the DA1-EOD1 pathway regulates organ sizes in lettuce predominantly by influencing cell proliferation rather than cell expansion, as illustrated by the increased cell number and decreased cell size in da1 and da1/eod1 mutants (FIG. 5). This aligns with the established role of DA1 as a key inhibitor of cell proliferation in Arabidopsis (FIG. 5; Li et al., 2008; Xia et al., 2013). These findings contribute significantly to our comprehension of the genetic factors that govern leaf size and overall organ development in plants.

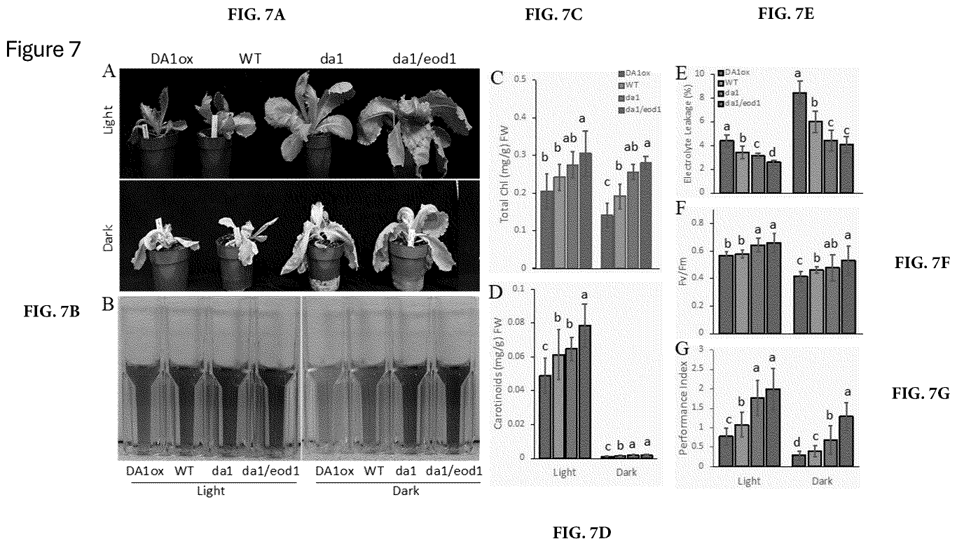

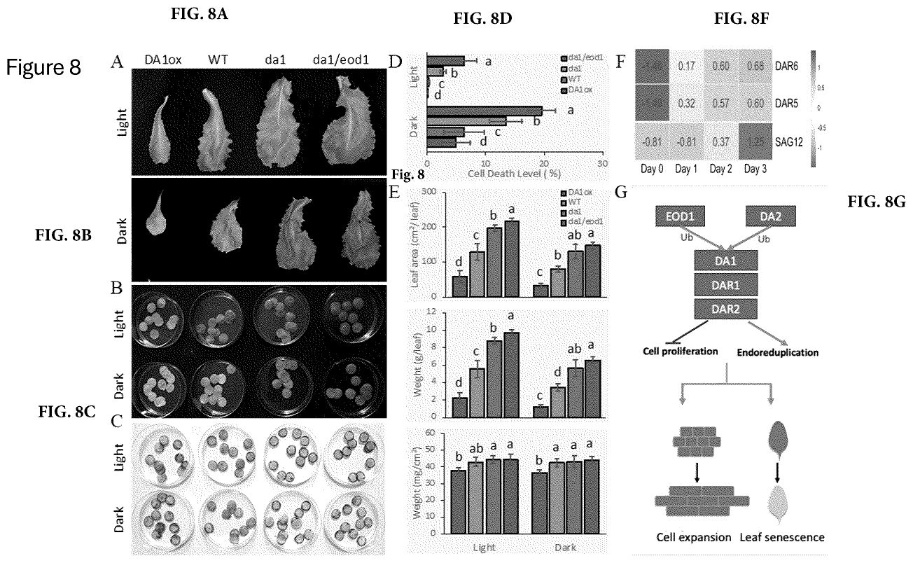

Chlorophylls are essential for capturing light energy and an indispensable pigment for photosynthesis. Chlorophyll b acts as an accessory pigment, aiding chlorophyll a by expanding the spectrum of captured light (Croce et al., 2014). An increase in chlorophyll content, especially Chlorophyll b, may enhance light absorption, potentially leading to increased biomass and yield in crops (Parry et al., 2014). While chlorophyll contents are not directly associated with organ size or cell proliferation, the improved photosynthesis due to higher chlorophyll levels may indirectly impact growth by providing the energy and substrates necessary for cell growth and division. Accordingly, a significant increase was observed in chlorophyll content as well as PI (abs), an indicator for plants photosynthetic performance, under both light and dark conditions (FIG. 7 and FIG. 8). The higher photosynthetic performance of da1/eod1 double mutant may provide more energy for faster cell growth, resulting in larger organ sizes in this mutant (FIG. 6, FIG. 7 and FIG. 8). Besides the enhanced cell proliferation, this could be another primary contributing factor leading to improved biomass in the da1 and da1/eod1 mutants.

### B.2. The Effect of DA1-EOD1 Module on Leaf Senescence

A previous study by Vanhaeren et al. (2017) demonstrated that ectopic expression of DA1 or EOD1 in Arabidopsis resulted in precocious leaf senescence, indicating their positive role in promoting leaf senescence. In this study, a notable stay-green phenotype was also observed in the da1/eod1 double mutant, primarily attributed to its increased chlorophyll content, indicating that the DA1-EOD1 module may regulate the chlorophyll synthesis or degradation.

The chlorophyll levels are determined by the balance between synthesis and degradation (Wang and Grimm, 2021). Chlorophyll degradation is a hallmark of leaf senescence, which is often accelerated by unfavorable conditions like severe shade or low light (Hortensteiner, 2006; Brouwer et al., 2012; Kuai et al., 2018; Li et al., 2023). Chlorophyll degradation begins with the conversion of Chl b to Chl a, regulated by two catabolic genes, NONYELLOW COLORING1 (NYC1) and HCAR (Kusaba et al., 2007; Horie et al., 2009; Sato et al., 2009; Meguro et al., 2011). Chl a is then degraded into pheophytin a, regulated by NON-YELLOWINGs/STAY GREENs (NYEs/SGRs) (Shimoda et al., 2016). The chlorophyll degradation pathway is conserved across plant species, and disruption of NYEs/SGRs leads to stay-green phenotypes in various plants (Jiang et al., 2007; Park et al., 2007; Barry et al., 2008; Zhou et al., 2011).

In this disclosure, it was observed that da1/eod1 double mutants significantly retain chlorophyll content, especially chlorophyll b (Chlb). Chlb plays a crucial role in plants' responses to environmental stress and in delaying the onset of senescence. Unlike chlorophyll a (Chla), which is central to photosynthesis, Chlb primarily facilitates light capture and energy transfer, especially in the LHCII type II complex under high light stress (Duffy et al., 2015). This function is key in dissipating excess energy, thus protecting the photosynthetic machinery. Elevated levels of Chlb may offer an advantage in countering stress-induced senescence (Duffy et al., 2015). Under stress, plants may increase Chlb levels to enhance light capture while concurrently protecting core photosynthetic components (Yang et al., 2022). For instance, in shaded environments, the synthesis of Chlb intensifies. However, during prolonged stressors, such as nutrient deficiencies, elevated Chlb levels could delay senescence, granting the plant more time to adapt (Pfündel et al., 1999). This is supported by a study in soybeans, which reported an increase in the Chlb to Chla ratio under low light, suggesting an adaptive mechanism to capture more light (Zhang et al., 2016). Thus, the increased Chlb levels in da1/eod1 double mutants could serve as a protective mechanism, potentially mitigating the impacts of both dark- and age-induced senescence by optimizing energy utilization and maintaining photosynthetic efficiency under adverse conditions.

Additionally, the transcriptomic analysis in this disclosure also indicated that DA1-related genes, including DAR5 and DAR6, are upregulated in the dark-induced senescent Arabidopsis leaves (FIG. 8F), posing a potential regulatory role of the DA1-EOD1 module in the dynamics of leaf senescence. According to definitions by Thomas (2014), stay-green mutants can be classified as functional and nonfunctional, depending on their ability to retain photosynthetic competence. Functional stay-green mutants maintain both leaf greenness and photosynthetic capacity for a longer duration, particularly under stress conditions, which can lead to increased crop yield. Because lettuce da1/eod1 mutant in this disclosure exhibited both retained photosynthetic capacity (as indicated by Psi) (FIG. 7H) and higher chlorophyll content under the dark stressful conditions, thus the da1/eod1 double mutants could be classified into functional stay-green mutants.

The precise molecular mechanisms of how DA1 and EOD1 regulate leaf senescence remain elusive. Nonetheless, recent studies have suggested a potential interaction between the DA1-EOD1 module and miRNA-mediated regulation of leaf senescence. miRNAs play an important role in leaf senescence regulatory pathway (Zhang et al., 2020). Notably, miR156 and miR164 are known to regulate leaf senescence by targeting SQUAMOSA PROMOTER BINDING PROTEIN-LIKE (SPL) and NAC (NAM, ATAF1/2, and CUC2) transcription factors such as ORESARA1 (ORE1), respectively (Jerome Jeyakumar et al., 2020; Li et al., 2013). Moreover, miR319 and miR396 have been shown to modulate leaf senescence by targeting TCP transcription factors (TCP14, TCP15, and TCP22) and growth-regulating factor (GRFs), respectively (Fang et al., 2021; Liebsch et al., 2020). Elevated levels of miR156 and miR164 were observed in the da1 and eod1 Arabidopsis mutants, suggesting these miRNAs might play a role in the delayed leaf senescence observed in these mutants (Kim et al., 2020). On the other hand, the reduced level of miR319 in the da1 mutant might contribute to its delayed leaf senescence (Kim et al., 2020). Additionally, in Brassica rapa, six specific miRNAs (br-miR164a, br-miR164b, br-miR164c, br-miR164d, br-miR360, and br-miR366) were found to target BrDAR6.1, BrDA1.4, and BrDA1.5. (Karamat et al., 2022). Collectively, these findings show that the DA1-EOD1 module may interact with miRNA pathways to regulate leaf senescence. However, a more in-depth investigation is needed to fully understand their role in this process.

### B.3. Potentials and Challenges of DA1-EOD1 Mutants in Controlled Environment Agriculture

Controlled Environment Agriculture (CEA), encompassing vertical farming and greenhouse hydroponics, has emerged as an alternative to conventional open-field production due to limited resources like irrigation water and farmland. CEA optimizes the utilization of resources such as water, fertilizer, and space, offering a sustainable solution for urban agricultural production (Specht et al., 2014; Oh and Lu, 2023; van Delden et al., 2021). Currently, lettuce is becoming the predominant crop species for CEA production. However, the expansion of CEA has been restrained by the high energy demands essential for maintaining precise environmental conditions (Avgoustaki and Xydis, 2020). As a full-sun vegetable, suboptimal light levels, particularly in vertical farming, pose a significant challenge. The limited breeding efforts for varieties suitable for indoor cultivation necessitate the development of lettuce with traits tailored to CEA production, which includes fast-growth, quick-turnover, high-yield, tolerance to controlled growth conditions, and improved postharvest quality.

In this context, the DA1-EOD1 module, known for their conserved roles regulating organ size in various plant species, offers as a promising innovation. The da1/eod1 lettuce mutants developed through CRISPR/Cas9 represents a significant breakthrough. These mutants exhibit rapid growth, larger leaf sizes, and a remarkable stay-green phenotype, indicating potential benefits such as increased yield, fast production turnover, tolerance to low light conditions, and improved postharvest quality. Harnessing the potential of the da1/eod1 double mutant could greatly transform lettuce production, mitigating supply chain vulnerabilities and meeting the growing global demand. This advancement is crucial not just for breeding superior lettuce varieties that offer both extended shelf life and rapid production cycles but also for strengthening global food security and adapting agricultural practices to changing climatic conditions.

The application of da1/eod1 mutants in lettuce offers a focused but limited viewpoint on the wider applicability of the DA1-EOD1 module across a spectrum of plant species and diverse agricultural settings. A deeper exploration and comparative study are necessary to unravel the complex molecular mechanisms, especially how the DA1-EOD1 module impacts leaf senescence under varying lighting conditions. Future research should encompass a thorough investigation of the DA1-EOD1 module across a diverse range of plant species, enriching our understanding of plant genetics and broadening the module's applicability in enhancing plant development and resilience. Such inquiries are crucial for integrating da1/eod1 traits into commercial lettuce varieties and evaluating their performance in real-world CEA scenarios, particularly considering the high light intensity requirements of lettuce and the associated energy costs in CEA. Delving into the unknown molecular mechanisms influencing the module's effect on leaf senescence under different light conditions, both during growth and post-harvest, is a vital research goal.

A balanced and nuanced research approach will not only extend the applicability of the findings but will also resonate with the burgeoning discourse on sustainable and resilient agricultural practices in an era of climate uncertainty. By exploring the intricate interactions between genes, plants, and the environment, and assessing the adaptability of da1/eod1 mutants to different environments, it is possible to uncover novel applications in CEA. This exploration is integral to addressing the urgent challenges of global food security and shaping a sustainable future for agriculture.

### C. Embodiment Examples

The study in this disclosure sheds light on the pivotal roles of the DA1-EOD1 module in governing diverse facets of plant growth, encompassing vegetative and reproductive growth, leaf senescence, and postharvest quality. These insights suggest that the strategic manipulation of the DA1-EOD1 module holds promising applications for refining plant breeding strategies and enhancing crop yield and productivity. When analyzed alongside existing studies, the implications of the research in this disclosure are profound, carrying considerable significance for Controlled Environment Agriculture (CEA) and the wider field of agriculture. Despite certain limitations, the delineated avenues for future research, signify promising prospects for addressing global food challenges and fostering advancements in plant genetics. Further exploration of the DA1-EOD1 module across different plant species and varied environmental contexts is essential to fully unravel both the conserved and species-specific regulatory mechanisms exerted by this module on plant growth and development.

Considering the effect of DA1 and EOD1 on plants regarding the aspects described above, in this disclosure are provided a gene editing system for DA1 and EOD genes and lettuce plant genetically modified in DA1 and EOD1 genes.

In some embodiments provide are one or more oligonucleotides comprising a nucleic acid sequence encoding a guide RNA for targeting (i.e., hybridizing to) a promoter or exon region of LsDA1 gene from LOC111914107 or LOC111876247 in lettuce (Lactuca sativa), or a nucleic acid sequence with at least 95% sequence identity thereof. The exemplary guide RNA targets an exon region of LsDA1 gene from LOC111914107, optionally exon 7 or exon 8 on chromosome 6, but other guide RNAs targeting other exon regions or promoter region of LsDA1 gene can be contemplated. The nucleic acid sequence of the exemplary guide RNA comprises SEQ ID NO:1 ATGCTGCTCTTAATGATGGG or SEQ ID NO:2 GCGTGGACTATGCCTCTCGG, optionally SEQ ID NO:1.

In some embodiments is provided oligonucleotides comprising a nucleic acid sequence encoding a guide RNA for targeting (i.e., hybridizing to) a promoter or exon region of LsEOD1 gene from LOC111885075, LOC111877487, or LOC111881104 in lettuce (Lactuca sativa), or a nucleic acid sequence with at least 95% sequence identity thereof. The exemplary guide RNA targets an exon region of LsEOD1 gene from LOC111885075, optionally exon 1 or exon 2 on chromosome 1, but other guide RNAs targeting other exon regions or promoter region of LsEOD1 gene can be contemplated. The nucleic acid sequence of the exemplary guide RNA comprises SEQ ID NO:3 TCATGGATAGGACCCGAATG or SEQ ID NO:4 GGATATGAATCGAAGTGCAT, optionally SEQ ID NO:3.

In other embodiments, plasmids comprising a nucleic acid sequence encoding any of SEQ ID NOs:1-4 for cloning (replication) or expression (transcription) are provided. In certain embodiments, the plasmid further comprises nucleic acid sequences encoding elements of a gene editing system, which comprises a nucleic acid sequence encoding at least one Cas endonuclease and nucleic acid sequences encoding selection markers, e.g., a fluorescent protein and an antibiotic resistance marker. In an exemplary embodiment, a nucleic acid sequence encoding a guide RNA targeting LsDA1 or nucleic acid sequences encoding guide RNAs targeting LsDA1 and LsEOD1 are cloned into the PHN-SpCas9-4×BsaI-GFP plasmid vector at BsaI sites through digestion and ligation. The plasmid vector comprises a T-DNA fragment flanked by left border (LB) sequence and right border (RB) sequence, and the gene editing system (i.e., gRNAs and Cas endonuclease) is cloned into the T-DNA multicloning site. The guide RNAs are fused with the tRNAs and driven by the Arabidopsis AtU6 RNA Pol III promoter. The PHN-SpCas9-4×BsaI-GFP vector contains a fused eGFP-NPTII under a dCsVMV promoter, a codon-optimized SpCas9 for Arabidopsis under parsley ubiquitin (PcUbi) promoter, and a sgRNA cloning cassette with two separate 2×BsaI sites, tRNAs and scaffold RNAs under the AtU6-26 promoter (ProAtU6-26-tRNA-2×BsaI-scaffold RNA-tRNA-2×BsaI-scaffold RNA polydT cassette, thus resulting in final PHN-SpCas9 CRISPR vector with 4×BsaI restriction site (PHN-SpCas9-4×BsaI-GFP) for convenient sgRNAs cloning). As an example of the tRNA, Trp-tRNA sequence is used, but theoretically, any amino acid-tRNA may be used for the same purpose.

In a more specific example, a gene editing system cloned into a T-DNA fragment in the plasmid vector comprises:
(i) a nucleic acid sequence encoding a guide RNA targeting LsDA1 gene, wherein the guide RNA comprises a spacer RNA of SEQ ID NO:1 or SEQ ID NO:2;
   alone or with
(ii) a nucleic acid sequence encoding a guide RNA targeting LsEOD1 gene, wherein the guide RNA comprises a spacer RNA of SEQ ID NO:3 or SEQ ID NO:4,
   wherein, when two nucleic acid sequences encoding two different guide RNAs are cloned, they are arranged consecutively with a nucleic acid sequence encoding a tRNA between them,
   wherein the nucleic acid sequence encoding a spacer RNA is flanked at its 5′-end by a nucleic acid sequence encoding a tRNA of SEQ ID NO:5 and at its 3′-end by a nucleic acid sequence encoding a scaffold RNA of SEQ ID NO:6, and
   wherein the nucleic acid sequence encoding the first tRNA is preceded at its 5′-end by an RNA polymerase III promoter, and the nucleic acid sequence encoding the last scaffold RNA is followed at its 3-end by a terminator sequence;
(iii) at least one nucleic acid sequence encoding a Cas endonuclease, wherein the Cas endonuclease is SpCas9, and wherein the nucleic acid sequence is preceded at its 5′-end by an RNA polymerase II promoter and followed at its 3′-end by a terminator sequence;
(iv) nucleic acid sequences encoding a fluorescent protein and an antibiotic resistance marker, wherein the nucleic acid sequences are preceded at its 5′-end by an RNA polymerase II promoter and followed at its 3-end by a terminator sequence.

In certain embodiments, each guide RNA and Cas endonuclease are located on the same vector or different vectors of the system. In other embodiments, instead of Cas9, other Cas endonucleases such as Cas12 can also be used.

In other embodiments, Agrobacterium comprising the plasmid described above is provided, and the Agrobacterium is optionally Agrobacterium tumefaciens.

In other embodiments, lettuce genetically modified using a gRNA comprising SEQ ID NO:1 or SEQ ID NO:2, which targets LsDa1 gene, is provided. In other embodiments, lettuce genetically modified using a gRNA comprising SEQ ID NO:1 or SEQ ID NO:2, which targets LsDa1 gene, and a gRNA comprising SEQ ID NO:3 or SEQ ID NO:4, which targets LsEod1 gene is provided.

Further, a method for genetic transformation to generate lettuce with improved organ size and postharvest quality is provided. Briefly, lettuce cotyledon explants from 10-day-old seedlings are incubated with Agrobacterium tumefaciens EHA105 harboring the appropriate vector and cultured on a callus-induction medium containing MS salts and vitamins, 1 mg/L 6-benzylaminopurine (6-BA), 0.1 mg/L α-naphthyacetic acid (NAA), 100 mg/L kanamycin, and 100 mg/L Timentin (ticarcillin/clavulanic acid) for three weeks and then transferred to shoot-induction medium containing MS salts and vitamins, 0.5 mg/L 6-BA, 0.01 mg/L NAA, 100 mg/L kanamycin, and 100 mg/L Timentin for three weeks. Root induction was performed using a medium containing 1/2 MS salts and vitamins, 50 mg/L kanamycin, and 100 mg/L Timentin.

Although Agrobacterium is used for introducing gene editing system into the plant cells as an exemplary method, other gene editing system delivery methods can be contemplated, e.g., plasmids or ribonucleoprotein complex transfection using PEG-CaCl₂ precipitation, electroporation, cell squeezing, sonoporation, optical transfection nanoparticles, magnetofection, and chemical or biological treatment as described in Sharma et al. (Sharma M, Bhushan S, Sharma D, Kaul S, Dhar M K. A Brief Review of Plant Cell Transfection, Gene Transcript Expression, and Genotypic Integration for Enhancing Compound Production. Methods Mol Biol. 2023; 2575:153-179).

### EXAMPLES

### Example 1. Materials and Methods

### 1.1 Plant Materials

Lettuce (Lactuca sativa, var “Salinas”) plants were grown under standard growth conditions with a 16-hour light/8-hour dark photoperiod at 22±1° C. and 60% relative humidity. Plants were grown in a soil mix containing perlite, vermiculite, and peat moss in a 1:1:1 ratio and watered as needed.

### 1.2. Vector Construction

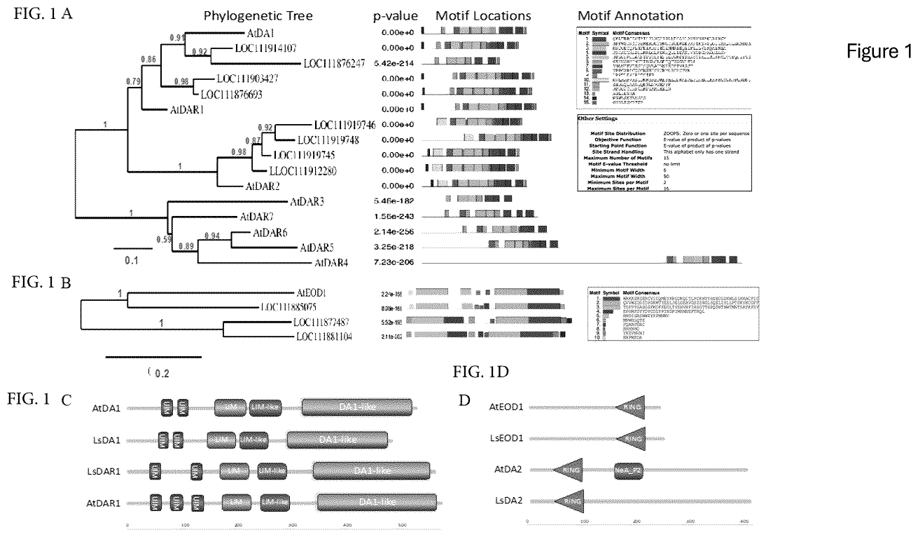

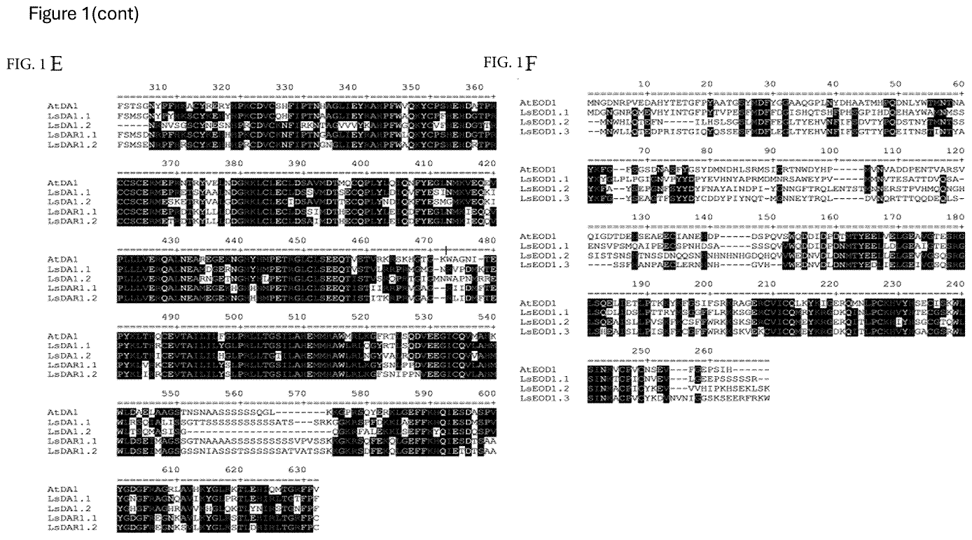

For DA1 overexpression: A pOX135 binary expression vector containing a fused eGFP-NPTII gene (for fluorescence and kanamycin selection) under a double-enhanced CsVMV (dCsVMV) promoter was used for overexpression of DA1 (Nguyen et al., 2021). The lettuce LsDA1 (XM_023909844) coding region with two BsaI restriction sites at 5′ and 3′ terminal end was synthesized and cloned into the pUC57-BsaI-Free cloning vector by Gene Universal Inc. (Newark, DE). The synthesized LsDA1 was released from this cloning vector through a BsaI digestion, and linearized LsDA1 was recovered, purified, and inserted into the pOX135 vector at the BsaI sites. The LsDA1 is driven by a 2×CaMV35S promoter containing an Omega enhancer from pGWB402 (Nakagawa et al., 2007) (FIG. 1).

For Gene Editing of LsDA1 and LsEOD1: The previously reported PHN-SpCas9-4×BsaI-GFP vector containing a fused eGFP-NPTII gene under a double-enhanced CsVMV (dCsVMV) promoter and a plant codon-optimized SpCas9 under a Parsley Ubiquitin promoter are used for this purpose (Nguyen et al., 2021). Two guided RNAs targeting LsDA1 (LOC111914107) or LsEOD1 (LOC111885075) were designed using the web tools CRISPR-P (crispr.hzau.edu.cn/CRISPR2/) (citations) and CRISPOR (crispor.tefor.net/) (citation). Only the guided RNAs with a higher editing efficiency and a lower off-target frequency predicted by both web tools were selected for synthesis through Gene Universal Inc. (Newark, DE). The guided RNAs targeting on LsDA1 or LsEOD1 were cloned into the same PHN-SpCas9-4×BsaI-GFP vector at BsaI sites through digestion and ligation. These guided RNAs were fused with the tRNAs and driven by the Arabidopsis AtU6 promoter (Lian et al, 2022).

### 1.3 Genetic Transformation

Lettuce tissue culture and Agrobacterium-mediated transformation were performed as described by Armas et al. (2017). Briefly, cotyledon explants from 10-day-old seedlings were incubated with Agrobacterium tumefaciens EHA105 harboring the appropriate vector and cultured on a callus-induction medium containing MS salts and vitamins, 1 mg/L 6-BA, 0.1 mg/L NAA, 100 mg/L kanamycin, and 100 mg/L Timentin for three weeks and then transferred to shoot-induction medium containing MS salts and vitamins, 0.5 mg/L 6-BA, 0.01 mg/L NAA, 100 mg/L kanamycin, and 100 mg/L Timentin for three weeks. Root induction was performed using a medium containing ½ MS salts and vitamins, 50 mg/L kanamycin, and 100 mg/L Timentin.

Visual detection of fluorescence in new roots and shoots for all seedlings was performed under a Leica MZFLIII fluorescent stereo microscope (Leica Microsystems, Germany) to initially screen transformed seedlings, followed by PCR genotyping.

### 1.4. PCR Genotyping

Genomic DNA was extracted from leaf tissues using the CTAB method (Dellaporta et al., 1983). PCR with primers for amplifying GFP was performed to detect the presence of the transgene. PCR was carried out with Q5 HotStart High-Fidelity DNA polymerase (New England BioLabs, Ipswich, MA) according to the manufacturer's instructions. Amplified DNA fragments were separated by electrophoresis on 1.2% (wt/v) agarose gels, stained with SYBR green (Thermo Scientific, Waltham, MA), and visualized with the Omega Lum™ Imaging system (Gel Company, San Francisco, CA). The primers used for GFP amplification are listed in Table 2.

### 1.5 Detection of Gene-Edited Mutations

Mutations were detected using the PCR Sanger sequencing method. Genomic DNA was isolated from the TO young seedling of each independent transgenic line, and PCR reactions were performed using a set of primers (Table Si) flanking two guided RNA sites in either LsDA1 or LsEOD1. PCR fragments were purified using Wizard SV Gel and PCR Clean-Up System kit following the manual's instructions prior to Sanger sequencing at Promega, Corp. (Madison, WI, USA). The chromatogram data from both mutants and wild type were uploaded to ICE platform (ice.synthego.com) for mutation detection.

### 1.6 Histological Analysis

To determine cell size and number, lettuce leaf and stem sections were prepared and stained using standard methods. For optimal results, immerse leaf samples in an FAA solution (10% formalin, 5% acetic acid, 50% ethanol) for 24 hours, followed by storage in 70% ethanol. To remove chlorophyll, place the samples in 70% ethanol, refreshing daily until leaves become pale, typically in 2-3 days. For clearing, immerse samples in 100% lactic acid until samples turn transparent. After rinsing in distilled water, stain the cleared leaves in 0.05% toluidine blue O for 1-5 minutes. Mount the stained samples on microscope slides using glycerol or water, pressing a cover slip atop to eliminate air bubbles. Examine the new blue-colored cell walls against a transparent background under a compound microscope.

### 1.7 Cell Death Detection

For cell death detection, lactophenol-trypan blue staining was employed. The trypan blue stock solution was prepared by combining 10 g of phenol, 10 mL each of glycerol, lactic acid, distilled water, and 0.02 g of trypan blue (Sigma, USA). This solution was diluted with 96% ethanol (1:2, v/v) to form the working solution. Lettuce leaf discs were submerged in the working solution, boiled for 1 minute, and incubated for 24 hours. Post-incubation, leaves were cleared in saturated chloral hydrate solution (1 kg chloral hydrate in 400 mL distilled water). Cell death quantification involved scanning fifteen leaves per treatment and analyzing trypan blue-stained areas using ImageJ's histogram tool (imagej.nih.gov/ij/). The ratio of stained to total leaf surface pixels was calculated to assess cell death.

### 1.8 RNA Extraction and RT-PCR Analysis

Total RNA was isolated from 100 mg of plant samples using RNAzol® RT RN190 (Molecular Research Center, Cincinnati, OH). cDNA was synthesized using the QuantiTect Reverse Transcription Kit (QIAGEN, Hilden, Germany) and diluted 25-fold. qRT-PCR reactions were composed of 4.5 μL diluted cDNA, 0.5 μL 10 μmol/L primers, and 5 μL BioRad SYBR® Green Master Mix (Thermo Fisher) and detected on a CFX96 real-time PCR system (BIO-RAD, Hercules, CA). Specific Primers used for detecting transcripts of organ size-related genes and leaf senescence-related genes were designed using Primer 3.0 at NCBI (ncbi.nlm.nih.gov/tools/primer-blast/) with the amplicon length ranging from 75 to 300 nt, and all primers for RT-PCR are listed in Table 2.

### 1.9 Chlorophyll Content Determination

Chlorophyll content was measured according to a modified method described by Lichtenthaler (1987). Transfer 1-2 treated leaves to a fresh Petri dish and rinse them 2-3 times with ultrapure water. Then, place 40-80 mg of these detached leaves in 2 mL centrifuge tubes, adding 1 mL of chlorophyll extraction solution (95% ethanol). Incubate these leaves in darkness at room temperature for 24 hours. Subsequently, measure the solution's absorbance at 665 nm and 649 nm, using the extraction solution as a control via BioTek SYNERGY H1 microplate reader (Highland Park, Winooski, VT, USA). Chlorophyll content can be derived using the given formulas:

$$Chlorophyll a ( μ g / mL ) = 13.95 \times {A}_{665} - 6.88 \times {A}_{649}$$

$$Chlorophyll b ( μ g / mL ) = 24.96 \times {A}_{649} - 7.32 \times {A}_{665}$$

$$Total chlorophyll ( mg / g FW ) = ( Chla + Chlb ) \times V / W$$

V: volume of extraction solution, 1 mL in this protocol; W: weight of freshly detached leaves.

### 1.10 Photosynthetic Measurements

Photosynthetic parameters were measured using OJIP to monitor photosystem II (PSII) performance and leaf senescence progression (Strasser et al., 2004). Chlorophyll fluorescence was recorded using a portable PEA fluorimeter (Hansatech Instruments, UK), and the resulting data was analyzed using the OJIP (LiCOR, Lincoln, NE., USA). Leaves were dark adapted at least 20 min before measuring; minimum (Fo) and maximum fluorescence (Fm) were obtained using LCF model according to the manufacturer's protocol. The variable fluorescence, Fv/Fm, was calculated as (Fm−Fo)/Fm.

### 1.11 Phenotypic Measurements

Images of leaves, flowers, and seeds captured under uniform lighting with a high-resolution camera. Images were analyzed by using an open-source image processing software Image J to measure their area, length, and width. Calibration was first performed by setting the scale using a known distance (Schindelin et al., 2012). Subsequently, the images were converted to 8-bit grayscale and thresholded to generate binary images, facilitating clear segmentation of plant organs. The “Analyze Particles” function was then used to identify and measure individual plant organs. Measurements were exported for further analysis and statistical comparison across genotypes and treatments.

### 1.12 Statistical Analysis

Statistical analysis and graph plotting were performed using R software. Data were assessed for normality and homogeneity of variances, and appropriate transformations were applied as needed. Depending on the data distribution, student's t-test, one-way ANOVA, or non-parametric tests were used for comparisons between groups. Significant differences were determined at the p<0.05 level. Graphs were generated using ggplot2 in R (Wickham, 2011).

### Example 2. Identification of DA1, and EOD1 Genes in Lettuce

By using Arabidopsis AtDA1 (AT1G19270) as a query for a blast analysis, two lettuce LsDA1s, LOC111914107 on chromosome 6 and LOC111876247 on chromosome 1 were identified. These two LsDA1s exhibit sequence similarity of 71.8% and 56.9% to AtDA1, respectively (FIG. 1A). Arabidopsis has one DA1 and seven DA1-related genes (DAR1 to DAR7). Further sequence analysis revealed that there are two DA1s, two DAR1s (LOC111876693 and LOC111903427), two DAR2s (LOC111919748 and LOC111919746), and two DAR2-like homologs (LOC111912280 and LOC111919745) in lettuce genome (FIGS. 1A and E). The phylogenetic analysis of all DA1s and DARs from Arabidopsis and lettuce showed that two LsDA1s (LOC111914107 and LOC111876247) and AtDA1 converged at a shared phylogenetic node, supported by a high bootstrap value of 91% (FIG. 1A). Notably, the shorter branch length for LsDA1s implies that it underwent fewer evolutionary changes since its divergence from a common ancestor with AtDA1 (FIGS. 1A and E). This phylogenetic relationship between LsDA1s and AtDA1 reinforces that they might share organ size regulatory functions across the two species. Interestingly, two LsDAR1s (LOC111876693 and LOC111903427) clustered more closely with the LsDA1 and AtDA1 than with AtDAR1, as supported by a bootstrap value of 86% (FIGS. 1A and E). This suggests that LsDAR1s may also influence lettuce organ size, possibly in a manner similar to LsDA1s and AtDA1.

In terms of protein structure, both LsDA1 and LsDAR1 possess two UIM motifs, a LIM domain, and the conserved DA1 peptidase motif (FIG. 1C). These structural components are indicative of their evolutionary protein sequence lineage.

Additionally, three lettuce homologs of AtEOD1 (AT1G19270): LOC111885075 on chromosome 1, LOC111877487 on chromosome 6, and LOC111881104 on chromosome 1, were also identified. They respectively exhibit 66.5%, 53.7%, and 54.4% sequence similarity with Arabidopsis EOD1 (FIGS. 1 B, D, and F). Notably, both LsEOD1 and LsDA2 E3 ligases have a RING domain that can interact with DA1, highlighting the functional and structural conservation of these E3 ligases (FIG. 1D).

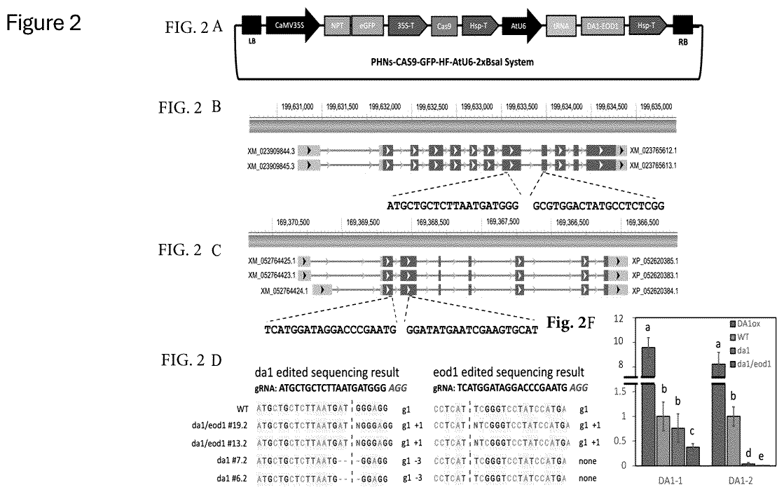

The sequence analysis showed that LOC111914107 and LOC111885075 are the most significantly similar to Arabidopsis AtDA1 and AtEOD1, and thus they were named as LsDA1 and LsEOD1, respectively. Interestingly, two splicing variants LsDA1, isoform X1 (XP_1023765612.1) and isoform X2 (XP_023765613.1), are derived from the same LsDA1 gene (LOC111914107), while LsEOD1 gene (LOC111885075) also gives rise to three protein isoforms, isoform X1 (XP_052620383.1), isoform X2 (XP_052620384.1), isoform X3 (XP_052620385.1). These isoforms likely result from alternative splicing events. Both LOC111914107 and LOC111885075 were selected for CRISPR editing targets (FIGS. 2 C and D).

### Example 3. Generation of Da1/Eod1 Double Mutant and LsDA1 Overexpression Transgenic Lettuce

In Arabidopsis, while single da1 or eod1 loss-of-function mutants slightly improve plant organ size, the da1/eod1 double mutants showed noticeably larger flowers and seeds (Li et al., 2008; Dong et al., 2017). To explore the effect of LsDA1 (LOC111914107) and LsEOD1 (LOC111885075) on lettuce organ size, lettuce da1 or/and eod1 mutant lines were generated using CRISPR/Cas9 gene editing. Two guided RNAs were designed for each gene to increase editing success (see methods) (FIGS. 2 B, C, and D). The first set of gRNAs targets the seventh and eighth exons of LsDA1 (LOC111914107 on chromosome 6) and the second pair of gRNAs target the first and second exons of LsEOD1 (LOC111885075 on chromosome 1) (FIG. 2B). Twenty independent transgenic lines carrying Cas9/sgRNAs transgene were obtained. Sanger sequencing of PCR fragments from these TO transgenic lines revealed that five TO transgenic lines have mutations in both genes and eight lines containing mutations in LsDA1 only. (FIG. 2E). The frameshift mutations in the DA1 and EOD1 caused premature stop codons that appeared before the first LIM domain for DA1 and E3 RING domain for EOD1 and before the conserved C-terminal region in DAR1a and DAR1b. The da1/eod1 mutants were backcrossed with Salinas wild type and then self-pollinated twice to obtain the BC1F3 generation for phenotypic analysis to eliminate the off-target mutations.

Next, two primer pairs were designed to evaluate whether the mutations in these mutants affect the transcription levels of LsDA1 in both the da1 single mutant and the da1/eod1 double mutant. The DA1-1 primers fully matched the exon region of at the C-Terminal end, while one of the DA1-2 primer set fully matched one of the guided RNAs (gRNAs) with the sequence 5′-ATGCTGCTCTTAATGATGGG-3′(SEQ ID NO: 1). Therefore, any changes, particularly at the 3-priming end of this sequence, will cause significant changes in the melting temperature during the qPCR reaction. This CRISPR editing in da1 and da1/eod1 mutants resulted in a single nucleotide insertion, altering the sequence to ATGCTGCTCTTAATGATTGGG in LsDA1 (SEQ ID NO: 27). Upon analysis, DA1-1 transcript levels remained consistent in the da1 single mutant, indicative of normal transcriptional activity. Interestingly, there was a noticeable decrease in DA1-1 expression in the da1/eod1 double mutant, which is likely due to the modified regulatory influence of the mutated eod1 on DA1. In contrast, DA1-2 transcript levels were significantly reduced in both the da1 single and da1/eod1 double mutants, a consequence of the insertion that compromised the sequence's specificity for allele-specific primer recognition (FIG. 2F).

Moreover, ten independent LsDA1 overexpression transgenic lines were also generated in the Salinas background (FIG. 2A). PCR genotyping confirmed the presence of T-DNA in nine out of these ten TO transgenic lines. The total transcripts of LsDA1 were significantly increased in the examined LsDA1 overexpression line (FIG. 2F).

### Example 4. The Effect of LsDA1 and LsEOD1 on Lettuce Vegetative Growth

During the vegetative phase, plants undergo a transition from juvenile to adult stages, which is marked by distinct alterations in leaf size, stem length, and hair distribution. This process, also known as heteroblasty, is regulated by a complex network of genes, including the DA1-EOD1 module (Raihan et al., 2021). Our study shows that the DA1-EOD1 module plays a key role in regulating leaf size and biomass accumulation in lettuce. By 10 weeks after germination (WPG), lettuce plants overexpressing LsDA1 (DA1ox) developed leaves that were approximately 40% smaller than those of the wild type (WT) counterparts (FIGS. 3 A and C). Accordingly, the fresh weight of DA1ox leaves was noticeably less than that of WT (FIGS. 3 A and D). In contrast, the da1/eod1 double mutant manifested a leaf area more than twice that of the WT, which is consistent with a 22% increase in fresh weight (FIG. 3D).

The trends became even more pronounced when the growth rate over time was examined. During the first 4-week vegetative growth after germination (WPG), DA1ox (DA1 overexpression) plants exhibited a slower leaf growth rate than wild-type plants, evidenced by a 19.0% reduction in leaf area compared to the WT during this growth period (FIGS. 3 B and C). In contrast, the da1 exhibited an 38.0% increase, while the da1/eod1 double mutant exhibited a striking 66.3% increase in leaf area relative to the WT during the same period (FIG. 3B; FIG. 4 A). After 6 weeks of growth, the da1 and da1/eod1 mutants demonstrated a 42.5% and 75.0% increase in leaf area relative to the WT, respectively. By 8 WPG, the leaf growth difference between the WT and mutants was more significant, reaching 81.8% and 138.0% for da1 or da1/eod1 mutants, respectively (FIGS. 3 A and B). In contrast, the growth of DA1ox plants consistently lagged behind over these growth periods, exhibiting reductions of 54.1% and 68.7% in leaf area relative to the WT at the 6- and 8-WPG, respectively. This divergent growth pattern was consistently noticeable from 4WPG to 10WPG (FIG. 3B), with a much faster leaf growth in da1/eod1 double mutant and a slower growth rate observed in DA1ox transgenic line compared to the WT (FIGS. 3 B and C).

Consistent with its leaf growth rate, the aboveground biomass of DA1ox plants was 36.4% lower than the WT (FIGS. 4 A, B, and C). In contrast, the aboveground biomass of da1 and da1/eod1 mutants exceeded the WT's by 37.5% and 70.2%, respectively (FIGS. 4 A, B, and C). In addition, root biomass followed a similar trend. DA1ox plants had a 37.5% reduction in root biomass compared to the WT, while the da1/eod1 double mutants exhibited a considerable 27.6% increase in root biomass relative to WT (FIG. 4D). The root biomass of da1 single mutant did not show significant change.

The influence of the DA1-EOD1 module extends to plant height as well. The time-course data on plant height revealed consistent differences between transgenic lines and wild type in their growth before bolting. The da1 and da1/eod1 mutant plants were consistently higher, while DA1ox plants were uniformly shorter than the wild type plants from 2 to 12 WPG. The largest difference was observed at 10WPG, with a 29.6% and 47.8% increase in plant height for the da1 and da1/eod1 mutants relative to the WT, respectively. In contrast, the plant height of DA1ox plants was 49.9% less compared to the wild type (FIG. 4E). These findings highlight the suppressive role of the DA1-EOD1 module in regulating lettuce growth.

### Example 5. Influence of LsDA1 and LsEOD1 on Lettuce Reproductive Growth

In addition to the inhibitory effect of LsDA1 and LsEOD1 on leaf growth, a noticeable impact of both genes in lettuce reproductive growth was observed, with agronomic traits including the development of flowers and seeds. The effect of both LsDA1 and LsEOD1 on flower size is pronounced. Flowers of DA1ox plants are 9.1% shorter compared to the WT plants, while the average individual flower length in the da1 and da1/eod1 mutants are about 8.1% and 11.5% longer than those of the WT plants, respectively (FIGS. 5 A and C). A similar trend was also observed in flower fresh weight. DA1ox flowers are 26.9% lighter than the WT, while the da1 and da1/eod1 mutants show 48.8% and 71.4% heavier compared to the WT (FIG. 5D).

In accordance with the flower size, alterations in seed size and seed weight were also observed in DA1 overexpression lines (DA1ox), da1, and da1/eod1 mutants. Seed area of DA1ox plants are 8.5% smaller and their 100-seed weight is 11.5% less than the WT. In contrast, da1, and da1/eod1 seeds are 10.8% and 23.2% larger in area than the WT, respectively (FIGS. 5 C, F, and G), and the 100-seed weight for da1 and da1/eod1 mutants are 10.6% and 31.3% more than the WT, respectively (FIGS. 5 C and G). Regarding seed yield per plant, DA1ox plants had a notable 54.5% lower compared to the WT, whereas average seed yield in da1 and da1/eod1 mutants are 46.5% and 99.5% higher than the WT, respectively (FIGS. 5 C and H).

The DA1ox plants bolted 1.3 days later and flowered 2.0 days on average later than the WT plants. In contrast, the da1 single mutant exhibited bolting 2.2 days earlier and flowering 0.3 days earlier. Additionally, the da1/eod1 double mutants exhibited a delay of 3.3 and in bolting and 1.2 days in flowering compared to the WT plants, respectively (FIG. 5E). This suggested that overexpressing the DA1 could potentially expedite the transition from vegetative to reproductive growth, while disrupting both DA1 and EOD1 could slow down this process. Collectively, these results suggested that both DA1 and EOD1 genes play a significant role in lettuce reproductive development and growth.

### Example 6. Regulation of Lettuce Organ Size by DA1-EOD1 Module Through Modifying Cell Proliferation and Cell Expansion

To understand the functional roles of the DA1-EOD1 module in modifying cell proliferation and/or cell expansion to regulate lettuce organ size, the leaf epidermal cell size and number were examined. Histological examinations of leaves from the da1, da1/eod1, DA1ox, and WT plants unveiled cellular differences among these genotypes. Specifically, DA1ox plants exhibited a 24.5% increase in epidermal cell size compared to the WT (FIGS. 6 A and B), whereas there was a decrease in the number of leaf epidermal cells by about 11.0% in DA1ox plants (FIGS. 6 A and C). In contrast to DA1ox, leaf epidermal cells of da1 and da1/eod1 mutants showed a decrease in size by approximately 19.6% and 37.1%, respectively, compared to those in the WT. However, the number of leaf epidermal cells in da1 and da1/eod1 mutants increased by around 19.4% and 54.7% compared to wide type (FIGS. 6 A and C). These findings suggest that loss of function in DA1 gene alone or in combination with EOD1 potentially leads to enhanced cell expansion but a reduction in cell proliferation.

Complementary to these detailed observations, qRT-PCR analyses have further validated our understanding. Two genes, CYCD3 and CDC27, key players in cell cycle regulation and progression, were examined as molecular markers of cell proliferation. CYCD3, a member of the cyclin D family, is instrumental in transitioning cells from the G1 to the S phase of the cell cycle, while CDC27 is part of the anaphase-promoting complex/cyclosome, crucial for the separation of sister chromatids during mitosis (Moon et al., 2004; de Freitas et al., 2013) On the other hand, EXPA1 and EXPB3 are involved in cell wall loosening and elongation, serving as indicators of cell expansion. EXPA1 encodes an expansin that disrupts hydrogen bonds between cell wall polysaccharides, facilitating turgor-driven cell extension. EXPB3, another expansin gene, likely plays a similar role (Wang et al., 2021). In the da1/eod1 mutants, there was a pronounced upregulation of CYCD3 and CDC27, indicative of an increased division rate, while these genes were downregulated in the DA1ox plants, suggesting a suppressive effect on cell cycle progression. Conversely, the EXPA1 and EXPB3 were markedly upregulated in DA1ox plants, denoting enhanced cell enlargement activity. In contrast, the da1 single mutant and da1/eod1 double mutants did not exhibit significant changes in these markers, except for a decrease in EXPA1 expression in the da1/eod1 double mutant. This pattern underscores DA1's role as an essential negative regulator of both cell proliferation and division and suggests a complex regulatory network controlling cell growth dynamics (FIG. 6D).

### Example 7. Regulation of Leaf Senescence by DA1-EOD1 Module in Lettuce

The da1/eod1 double mutant unexpectedly exhibited a distinct “stay-green” phenotype, a characteristic not previously reported (FIGS. 7A and 8A). Six-week-old plants, each genotype was placed in either light or dark conditions for 5 days. Next was measured how the physiological parameters associated with leaf senescence and photosynthesis were altered in DA1ox lines and the da1 and da1/eod1 mutants.

Chlorophyll content or chlorophyll degradation is a critical indicator for leaf senesce and photosynthetic capacity, particularly under stressful conditions. The DA1ox line exhibited a decrease in the total chlorophyll content by 15.2% in light and 26.2% in dark conditions compared to the wild type (FIGS. 7 A, B, and C). The da1 and da1/eod1 mutants demonstrated a significant increase in chlorophyll levels under both light and dark conditions. They showed increases of approximately 13.6% and 26.2% under light, and even more notable increments of 33.7% and 46.2% under dark conditions, respectively. The Fv/Fm ratio, an indicator of the maximum quantum efficiency of Photosystem II, also improved in the mutants, with the da1 and da1/eod1 showing increases of around 12.0% and 14.6% under light, and 3.1% and 14.9% under dark conditions (FIG. 7F). Furthermore, the Performance Indexes (PI (abs)), which indicate the overall photosynthetic performance, were substantially higher in da1 and da1/eod1 mutants under both light (64.0% and 85.2%) and dark conditions (74.5% and 226.5%), highlighting the enhanced photosynthetic activity in these mutants (FIG. 7G).

Similar to what was described in the previous section, the da1/eod1 mutant exhibited a 69.0% larger in leaf size compared to the wild type under the light condition after five-day treatments. This difference is more pronounced when these plants were placed in the dark for 5 days. The leaf area of da1/eod1 mutant exhibited an 83.8% larger compared to the wild type under dark conditions, which probably contributed to the higher chlorophyll content and photosynthetic potentials (FIG. 7A; FIGS. 8 A and E). In accordance with leaf area, fresh leaf weight also showed notable gains. Under light conditions, the increases were 58.3% and 74.8% for the da1 and da1/eod1 mutants. In dark conditions, the increases were even more pronounced, at 65.6% for da1 and 90.6% for da1/eod1 mutants, respectively (FIG. 8E). The fresh leaf weight per unit area showed slight increases, indicating denser or thicker leaves in these mutants (FIGS. 8 A and E).

Carotenoids, known for their antioxidant properties, are essential and beneficial nutrients for human health. In our study, compared to the wild type, the total carotenoids were found to increase by 5.3% and 27.9% in the da1 and da1/eod1 mutants under light conditions, respectively. Under the dark conditions, this difference is more notable, with 44.0% and 48.0% higher for the da1 and da1/eod1 mutants relative to the wild type, respectively. Conversely, the DA1ox line exhibited a decrease in carotenoid levels, showing reductions of 20.6% under light and 21.6% under dark conditions. Notably, the total carotenoids in all genotypes dramatically plummeted after five days of dark treatment. These results suggested that biosynthesis and stability of carotenoids are highly sensitive to light availability, and their rapid degradation in darkness could significantly affect the nutritional quality of postharvest lettuce (FIG. 7D).

Ion leakage, a marker of cell membrane integrity, was reduced in the da1 and da1/eod1 mutants by 8.4% and 24.2% under light conditions, and 26.4% and 32.4% under dark conditions, respectively. This suggests a better cellular membrane integrity in these mutants (FIG. 7E). An intact cellular membrane acts as a barrier for large molecules, including the trypan blue dye. Consequently, only dead or damaged cells can absorb this dye, resulting in a blue stain. Trypan blue staining showed significantly lower levels of cell death in these mutants compared to the wild type (Kerschbauma et al., 2021), while no notable difference in the cell viability was observed between the wild type and the da1/eod1 double mutant under the light conditions, under dark conditions, the wild type exhibited more dead or damaged cells, with only 63.0% of the levels seen in the da1/eod1 mutant (FIGS. 8 C and D). In contrast, the DA1ox line displayed an increase in dead or damaged cells, up to 129.4% compared to the wild type. These results indicated that DA1 and EOD1 may promote cell death under stressful conditions, thereby accelerating leaf senescence procession (FIGS. 8 A, B, C, and D).

TABLE 1
Nucleic acid sequences for the gene editing system

| SEQ ID | | | |
|---|---|---|---|
| NO. | RNA | Sequence | Note |
| 1 | spacer RNA targeting LsDA1 | ATGCTGCTCTTAATGATGGG | From exon 7, |
| | | | LOC111914107 |
| 2 | spacer RNA targeting LsDA1 | GCGTGGACTATGCCTCTCGG | From exon 8, |
| | | | LOC111914107 |
| 3 | spacer RNA targeting | TCATGGATAGGACCCGAATG | From exon 1, |
| | LsEOD1 | | LOC111885075, |
| 4 | spacer RNA targeting | GGATATGAATCGAAGTGCAT | From exon 2, |
| | LsEOD1 | | LOC111885075, |
| 5 | tRNA | AACAAAGCACCAGTGGTCTAGTGGTA | 5′ flanking sequence |
| | | GAATAGTACCCTGCCACGGTACAGAC | of the gRNA |
| | | CCGGGTTCGATTCCCGGCTGGTGCA | |
| 6 | Scaffold RNA sequence in the | GTTTTAGAGCTAGAAATAGCAAGTTA | 3′ sequence in the |
| | gRNA | AAATAAGGCTAGTCCGTTATCAACTT | gRNA |
| | | GAAAAAGTGGCACCGAGTCGGTGC | |

TABLE 2
Primers for genotyping and qRT-PCR

| SEQ | | | |
|---|---|---|---|
| ID | Primer | | |
| NO. | Name | Sequence | Note |
| 11 | GFP_ | ACAAGTTCAGCGTGTCCG | Genotyping |
| | detector- | | |
| | F | | |
| 12 | GFP_ | TCACCTTGATGCCGTTCT | Genotyping |
| | detector- | | |
| | R | | |
| 13 | AtU6_F5 | GCCCCTGGGAATCTGAAAG | Genotyping |
| 14 | PHNCAS9_ | TAAGTTGGGTAACGCCAGGG | Genotyping |
| | Bone_R2 | | |
| 15 | LsDA1-1-F | ACCGTCAGCACAGTTTTGAG | qRT-PCR |
| 16 | LsDA1-1-R | CCAGTCAGCAACCTTGGAAG | qRT-PCR |
| 17 | LsDA1-2-F | ATCGAGTATAGGGCCCATCC | qRT-PCR |
| 18 | LsDA1-2-R | AGGCATAGCTTCCTCCCATC | qRT-PCR |
| 19 | LsCYCD3-F | AGGGTAGCAGTTGTTGGAGC | qRT-PCR |
| 20 | LsCYCD3-R | AAATCGACCAACCACGACCA | qRT-PCR |
| 21 | LsCDC27-F | GACAGCTGGCAGAGAGTGTT | qRT-PCR |
| 22 | LsCDC27-R | GTAGCAGCAGCATTTCCAGC | qRT-PCR |
| 23 | LsEXPB3-F | TGGGCCGATTATGAACTCCG | qRT-PCR |
| 24 | LsEXPB3-R | CGCGCTCTAAATGGCTTCAC | qRT-PCR |
| 25 | LsEXPA1-F | TCAATTGCGTCCTCCGTCAA | qRT-PCR |
| 26 | LsEXPA1-R | CACAAGCACCACCCATTGTG | qRT-PCR |

>SpCas9, 4104 bp
(SEQ ID NO: 7)
atggataagaagtactctatcggactcgatatcggaactaactctgtgggatgggctgtgatcaccgatgagtacaaggtgccatctaagaa
gttcaaggttctcggaaacaccgataggcactctatcaagaaaaaccttatcggtgctctcctcttcgattctggtgaaactgctgaggctacc
agactcaagagaaccgctagaagaaggtacaccagaagaaagaacaggatctgctacctccaagagatcttctctaacgagatggctaaa
gtggatgattcattcttccacaggctcgaagagtcattcctcgtggaagaagataagaagcacgagaggcaccctatcttcggaaacatcgtt
gatgaggtggcataccacgagaagtaccctactatctaccacctcagaaagaagctcgttgattctactgataaggctgatctcaggctcatc
tacctcgctctcgctcacatgatcaagttcagaggacacttcctcatcgagggtgatctcaaccctgataactctgatgtggataagttgttcat
ccagctcgtgcagacctacaaccagcttttcgaagagaaccctatcaacgcttcaggtgtggatgctaaggctatcctctctgctaggctctct
aagtcaagaaggcttgagaacctcattgctcagctccctggtgagaagaagaacggacttttcggaaacttgatcgctctctctctcggactc
acccctaacttcaagtctaacttcgatctcgctgaggatgcaaagctccagctctcaaaggatacctacgatgatgatctcgataacctcctcg
ctcagatcggagatcagtacgctgatttgttcctcgctgctaagaacctctctgatgctatcctcctcagtgatatcctcagagtgaacaccgag
atcaccaaggctccactctcagcttctatgatcaagagatacgatgagcaccaccaggatctcacacttctcaaggctcttgttagacagcag
ctcccagagaagtacaaagagattttcttcgatcagtctaagaacggatacgctggttacatcgatggtggtgcatctcaagaagagttctaca
agttcatcaagcctatcctcgagaagatggatggaaccgaggaactcctcgtgaagctcaatagagaggatcttctcagaaagcagaggac
cttcgataacggatctatccctcatcagatccacctcggagagttgcacgctatccttagaaggcaagaggatttctacccattcctcaaggat
aacagggaaaagattgagaagattctcaccttcagaatcccttactacgtgggacctctcgctagaggaaactcaagattcgcttggatgacc
agaaagtctgaggaaaccatcaccccttggaacttcgaagaggtggtggataagggtgctagtgctcagtctttcatcgagaggatgacca
acttcgataagaaccttccaaacgagaaggtgctccctaagcactctttgctctacgagtacttcaccgtgtacaacgagttgaccaaggttaa
gtacgtgaccgagggaatgaggaagcctgcttttttgtcaggtgagcaaaagaaggctatcgttgatctcttgttcaagaccaacagaaaggt
gaccgtgaagcagctcaaagaggattacttcaagaaaatcgagtgcttcgattcagttgagatttctggtgttgaggataggttcaacgcatct
ctcggaacctaccacgatctcctcaagatcattaaggataaggatttcttggataacgaggaaaacgaggatatcttggaggatatcgttctta
ccctcaccctctttgaagatagagagatgattgaagaaaggctcaagacctacgctcatctcttcgatgataaggtgatgaagcagttgaaga
gaagaagatacactggttggggaaggctctcaagaaagctcattaacggaatcagggataagcagtctggaaagacaatccttgatttcctc
aagtctgatggattcgctaacagagccttcgcggctctcatcgctgatgattctctcacctttaaagaggatatccagaaggctcaggtttcag
gacagggtgatagtctccatgagcatatcgctaacctcgctggatctcctgcaatcaagaagggaatcctccagactgtgaaggttgtggat
gagttggtgaaggtgatgggaaggcataagcctgagaacatcgtgatcgaaatggctagagagaaccagaccactcagaagggacaga
agaactctagggaaaggatgaagaggatcgaggaaggtatcaaagagcttggatctcagatcctcaaagagcaccctgttgagaacactc
agctccagaatgagaagctctacctctactacctccagaacggaagggatatgtatgtggatcaagagttggatatcaacaggctctctgatt
acgatgttgatcatatcgtgccacagtcattcttgaaggatgattctatcgataacaaggtgctcaccaggtctgataagaacaggggtaaga
gtgataacgtgccaagtgaagaggttgtgaagaaaatgaagaactattggaggcagctcctcaacgctaagctcatcactcagagaaagtt
cgataacttgactaaggctgagaggggaggactctctgaattggataaggcaggattcatcaagaggcagcttgtggaaaccaggcagat
cactaagcacgttgcacagatcctcgattctaggatgaacaccaagtacgatgagaacgataagttgatcagggaagtgaaggttatcaccc
tcaagtcaaagctcgtgtctgatttcagaaaggatttccaattctacaaggtgagggaaatcaacaactaccaccacgctcacgatgcttacct
taacgctgttgttggaaccgctctcatcaagaagtatcctaagctcgagtcagagttcgtgtacggtgattacaaggtgtacgatgtgaggaa
gatgatcgctaagtctgagcaagagatcggaaaggctaccgctaagtatttcttctactctaacatcatgaatttcttcaagaccgagattaccc
tcgctaacggtgagatcagaaagaggccactcatcgagacaaacggtgaaacaggtgagatcgtgtgggataagggaagggatttcgcta
ccgttagaaaggtgctctctatgccacaggtgaacatcgttaagaaaaccgaggtgcagaccggtggattctctaaagagtctatcctcccta
agaggaactctgataagctcattgctaggaagaaggattgggaccctaagaaatacggtggtttcgattctcctaccgtggcttactctgttct
cgttgtggctaaggttgagaagggaaagagtaagaagctcaagtctgttaaggaacttctcggaatcactatcatggaaaggtcatctttcga
gaagaacccaatcgatttcctcgaggctaagggatacaaagaggttaagaaggatctcatcatcaagctcccaaagtactcactcttcgaact
cgagaacggtagaaagaggatgctcgcttctgctggtgagcttcaaaagggaaacgagcttgctctcccatctaagtacgttaactttctttac
ctcgcttctcactacgagaagttgaagggatctccagaagataacgagcagaagcaacttttcgttgagcagcacaagcactacttggatga
gatcatcgagcagatctctgagttctctaaaagggtgatcctcgctgatgcaaacctcgataaggtgttgtctgcttacaacaagcacagagat
aagcctatcagggaacaggcagagaacatcatccatctcttcacccttaccaacctcggtgctcctgctgctttcaagtacttcgatacaacca
tcgataggaagagatacacctctaccaaagaagtgctcgatgctaccctcatccatcagtctatcactggactctacgagactaggatcgatc
tctcacagctcggtggtgat
>PcUbi Promoter, 963 bp
(SEQ ID NO: 8)
Ggatatgaatataggcatatccgtatccgaattatccgtttgacagctagcaacgattgtacaattgcttctttaaaaaaggaagaaagaaaga
aagaaaagaatcaacatcagcgttaacaaacggccccgttacggcccaaacggtcatatagagtaacggcgttaagcgttgaaagactcct
atcgaaatacgtaaccgcaaacgtgtcatagtcagatcccctcttccttcaccgcctcaaacacaaaaataatcttctacagcctatatatacaa
cccccccttctatctctcctttctcacaattcatcatctttctttctctacccccaattttaagaaatcctctcttctcctcttcattttcaaggtaaatctc
tctctctctctctctctctgttattccttgttttaattaggtatgtattattgctagtttgttaatctgcttatcttatgtatgccttatgtgaatatcttta
tcttgttcatctcatccgtttagaagctataaatttgttgatttgactgtgtatctacacgtggttatgtttatatctaatcagatatgaatttcttcatatt
gttgcgtttgtgtgtaccaatccgaaatcgttgatttttttcatttaatcgtgtagctaattgtacgtatacatatggatctacgtatcaattgttcatctgt
ttgtgtttgtatgtatacagatctgaaaacatcacttctctcatctgattgtgttgttacatacatagatatagatctgttatatcattttttttattaattg
tgtatatatatatgtgcatagatctggattacatgattgtgattatttacatgattttgttatttacgtatgtatatatgtagatctggactttttggagttg
ttgacttgattgtatttgtgtgtgtatatgtgtgttctgatcttgatatgttatgtatgtgcagc
>dCMV-NPTII-eGFP-35sTer, 2614 bp
(SEQ ID NO: 9)
ttcgggggatctggattttagtactggattttggttttaggaattagaaattttattgatagaagtattttacaaatacaaatacatactaagggtttct
tatatgctcaacacatgagcgaaaccctataggaaccctaattcccttatctgggaactactcacacattattatggagaaactcgagcttgcat
gccaattctagagcggccgcttatcagaagaactcgtcaagaaggcgatagaaggcgatgcgctgcgaatcgggagcggcgataccgta
aagcacgaggaagcggtcagcccattcgccgccaagctcttcagcaatatcacgggtagccaacgctatgtcctgatagcggtccgccac
acccagccggccacagtcgatgaatccagaaaagcggccattttccaccatgatattcggcaagcaggcatcgccatgggtcacgacgag
atcctcgccgtcgggcatgctcgccttgagcctggcgaacagttcggctggcgcgagcccctgatgctcttcgtccagatcatcctgatcga
caagaccggcttccatccgagtacgtgctcgctcgatgcgatgtttcgcttggtggtcgaatgggcaggtagccggatcaagcgtatgcag
ccgccgcattgcatcagccatgatggatactttctcggcaggagcaaggtgagatgacaggagatcctgccccggcacttcgcccaatagc
agccagtcccttcccgcttcagtgacaacgtcgagcacagctgcgcaaggaacgcccgtcgtggccagccacgatagccgcgctgcctc
gtcttgcagttcattcagggcaccggacaggtcggtcttgacaaaaagaaccgggcgcccctgcgctgacagccggaacacggcggcat
cagagcagccgattgtctgttgtgcccagtcatagccgaatagcctctccacccaagcggccggagaacctgcgtgcaatccatcaccatg
atggtgatgatgatggccctgcagtcccttgtacagctcgtccatgccgagagtgatcccggcggcggtcacgaactccagcaggaccatg
tgatcgcgcttctcgttggggtctttgctcagggcggactgggtgctcaggtagtggttgtcgggcagcagcacggggccgtcgccgatgg
gggtgttctgctggtagtggtcggcgagctgcacgctgccgtcctcgatgttgtggcggatcttgaagttcaccttgatgccgttcttctgcttg
tcggccatgatatagacgttgtggctgttgtagttgtactccagcttgtgccccaggatgttgccgtcctccttgaagtcgatgcccttcagctc
gatgcggttcaccagggtgtcgccctcgaacttcacctcggcgcgggtcttgtagttgccgtcgtccttgaagaagatggtgcgctcctgga
cgtagccttcgggcatggcggacttgaagaagtcgtgctgcttcatgtggtcggggtagcggctgaagcactgcacgccgtaggtcaggg
tggtcacgagggtgggccagggcacgggcagcttgccggtggtgcagatgaacttcagggtcagcttgccgtaggtggcatcgccctcg
ccctcgccggacacgctgaacttgtggccgtttacgtcgccgtccagctcgaccaggatgggcaccaccccggtgaacagctcctcgccc
ttgctcaccatggtggcgaccggtacccggggatccacaaacttacaaatttctctgaagttgtatcctcagtacttcaaagaaaatagcttac
accaaattttttcttgttttcacaaatgccgaacttggttccttatataggaaaactcaagggcaaaaatgacacggaaaaatataaaaggataa
gtagtgggggataagattcctttgtgataaggttactttccgcccttacattttccaccttacatgtgtcctctatgtctctttcacaatcaccgacct
tatcttcttcttttcattgttgtcgtcagtgcttacgtcttcaagattcttttcttcgcctggttcttctttttcaatttctacgtattcttcttcgtattct
ggcagtataggatcttgtatctgtacattcttcatttttgaacataggttgcatatgtgccgcatattgatctgcttcttgctgagctcacataatacttcca
tagtttttcccgtaaacattggattcttgatgctacatcttggataattaccttctgcagcccttacattttccaccttacatgtgtcctctatgtctcttt
cacaatcaccgaccttatcttcttcttttcattgttgtcgtcagtgcttacgtcttcaagattcttttcttcgcctggttcttctttttcaatttctacgtatt
cttcttcgtattctggcagtataggatcttgtatctgtacattcttcatttttgaacataggttgcatatgtgccgcatattgatctgcttcttgctgagc
tcacataatacttccatagtttttcccgtaaacattggattcttgatgctacatcttggataattaccttctg

### REFERENCES

Ampim P A Y, Obeng E, Olvera-Gonzalez E (2022) Indoor Vegetable Production: An Alternative Approach to Increasing Cultivation. Plants-Basel 11 (21).
Andriankaja M, Dhondt S, De Bodt S, Vanhaeren H, Coppens F, De Milde L, Miihlenbock P, Skirycz A, Gonzalez N, Beemster G T, Inze D (2012). Exit from proliferation during leaf development in Arabidopsis thaliana: a not-so-gradual process. Developmental cell, 22(1):64-78.
Armas I, Pogrebnyak N, Raskin I (2017). A rapid and efficient in vitro regeneration system for lettuce (Lactuca sativa L.). Plant Methods, 13(1):1-9.
Barry C S, McQuinn R P, Chung M Y, Besuden A, Giovannoni J J (2008) Amino acid substitutions in homologs of the STAY-GREEN protein are responsible for the green-flesh and chlorophyll retainer mutations of tomato and pepper. Plant Physiol 147 (1):179-187.
Brouwer B, Ziolkowska A, Bagard M, Keech O, Gardestrom P (2012) The impact of light intensity on shade-induced leaf senescence. Plant Cell Environ 35 (6):1084-1098. doi:10.1111/j.1365-3040.2011.02474.x
Cai X, Sun X, Xu C, Sun H, Wang X, Ge C, Zhang Z, Wang Q, Fei Z, Jiao C, Wang Q (2021). Genomic analyses provide insights into spinach domestication and the genetic basis of agronomic traits. Nature Communications, 12(1):7246.
Croce R, Van Amerongen H (2014). Natural strategies for photosynthetic light harvesting. Nature chemical biology, 10(7):492-501.
Depuydt S, Van Praet S, Nelissen H, Vanholme B, Vereecke D (2016). How plant hormones and their interactions affect cell growth. Molecular cell biology of the growth and differentiation of plant cells, 174.
Deshaies R J, Joazeiro C A (2009). RING domain E3 ubiquitin ligases. Annual review of biochemistry, 78:399-434.
de Freitas Lima M, Eloy N B, de Siqueira J A, Inze D, Hemerly A S, Ferreira P C (2017). Molecular mechanisms of biomass increase in plants. Biotechnology Research and Innovation, 1(1):14-25.
Dong H, Dumenil J, Lu F H, Na L, Vanhaeren H, Naumann C, Klecker M, Prior R, Smith C, McKenzie N, Saalbach G (2017). Ubiquitylation activates a peptidase that promotes cleavage and destabilization of its activating E3 ligases and diverse growth regulatory proteins to limit cell proliferation in Arabidopsis. Genes & development, 31(2):197-208.
Du L, Li N, Chen L, Xu Y, Li Y, Zhang Y, Li C, Li Y (2014). The ubiquitin receptor DA1 regulates seed and organ size by modulating the stability of the ubiquitin-specific protease UBP15/SOD2 in Arabidopsis. The Plant Cell, 26(2):665-77.
Duffy C D, Ruban A V (2015). Dissipative pathways in the photosystem-II antenna in plants. Journal of Photochemistry and Photobiology B: Biology, 152:215-26.
Dutta M, Mali S, Raturi V, Zinta G (2023). Transcriptional and Post-transcriptional Regulation of Tuberization in Potato (Solanum tuberosum L.). Journal of Plant Growth Regulation, 1-24.
Fang Y, Zheng Y, Lu W, Li J, Duan Y, Zhang S, Wang Y (2021). Roles of miR319-regulated TCPs in plant development and response to abiotic stress. The Crop Journal, 9(1):17-28.
Gong P, Demuynck K, De Block J, Aesaert S, Coussens G, Pauwels L, Inze D, Nelissen H (2022). Modulation of the DA1 pathway in maize shows that translatability of information from Arabidopsis to crops is complex. Plant Science:111295.
Gonzalez N, Vanhaeren H, Inze D (2012). Leaf size control: complex coordination of cell division and expansion. Trends in plant science, 17(6):332-40.
Hicke L, Schubert H L, Hill C P (2005). Ubiquitin-binding domains. Nature reviews Molecular cell biology, 6(8):610-21.
Horie Y, Ito H, Kusaba M, Tanaka R, Tanaka A (2009) Participation of Chlorophyll b Reductase in the Initial Step of the Degradation of Light-harvesting Chlorophyll a/b-Protein Complexes in Arabidopsis. J Biol Chem 284 (26):17449-17456.
Hortensteiner S (2006) Chlorophyll degradation during senescence. Annu Rev Plant Biol 57:55-77. doi:10.1146/annurev.arplant.57.032905.105212
Jerome Jeyakumar J M, Ali A, Wang W M, Thiruvengadam M (2020). Characterizing the role of the miR156-SPL network in plant development and stress response. Plants, 9(9):1206.
Jiang H W, Li M R, Liang N B, Yan H B, Wei Y L, Xu X, Liu J F, Xu Z, Chen F, Wu G J (2007) Molecular cloning and function analysis of the stay green gene in rice. Plant J 52 (2):197-209.
Johnson K, Lenhard M (2011). Genetic control of plant organ growth. New Phytologist, 191(2):319-33.
Kuai B, Chen J Y, Hortensteiner S (2018) The biochemistry and molecular biology of chlorophyll breakdown. J Exp Bot 69 (4):751-767.
Kusaba M, Ito H, Morita R, Iida S, Sato Y, Fujimoto M, Kawasaki S, Tanaka R, Hirochika H, Nishimura M, Tanaka A (2007) Rice NON-YELLOW COLORING1 is involved in light-harvesting complex II and grana degradation during leaf senescence. Plant Cell 19 (4):1362-1375.
Li A, Zhou Y, Jin C, Song W, Chen C, Wang C (2013). LaAP2L1, a heterosis-associated AP2/EREBP transcription factor of Larix, increases organ size and final biomass by affecting cell proliferation in Arabidopsis. Plant and cell physiology, 54(11):1822-36.
Li Z, Peng J, Wen X, Guo H (2013). Ethylene-insensitive3 is a senescence-associated gene that accelerates age-dependent leaf senescence by directly repressing miR164 transcription in Arabidopsis. The Plant Cell, 25(9):3311-28.
Li Y, Zheng L, Corke F, Smith C, Bevan M W (2008). Control of final seed and organ size by the DA1 gene family in Arabidopsis thaliana. Genes & Development, 22(10):1331-6.
Li Z, Zhao T, Liu J, Li H Y, Liu B (2023) Shade-Induced Leaf Senescence in Plants. Plants-Basel 12 (7).
Lian Z, Nguyen C D, Liu L, Wang G, Chen J, Wang S, Yi G, Wilson S, Ozias-Akins P, Gong H, Huo H (2022). Application of developmental regulators to improve in planta or in vitro transformation in plants. Plant Biotechnology Journal, 20(8):1622-35.
Liebsch D, Palatnik J F (2020). MicroRNA miR396, GRF transcription factors and GIF co-regulators: a conserved plant growth regulatory module with potential for breeding and biotechnology. Current opinion in plant biology, 53:31-42.
Liu H, Li H, Hao C, Wang K, Wang Y, Qin L, An D, Li T, Zhang X (2020). TaDA1, a conserved negative regulator of kernel size, has an additive effect with TaGW2 in common wheat (Triticum aestivum L.). Plant Biotechnology Journal, 18(5):1330-42.
Managa M G, Tinyani P P, Senyolo G M, Soundy P, Sultanbawa Y, Sivakumar D (2018) Impact of transportation, storage, and retail shelf conditions on lettuce quality and phytonutrients losses in the supply chain. Food Sci Nutr 6 (6):1527-1536. doi:10.1002/fsn3.685
Mao H, Sun S, Yao J, Wang C, Yu S, Xu C, Li X, Zhang Q (2010). Linking differential domain functions of the GS3 protein to natural variation of grain size in rice. Proceedings of the National Academy of Sciences:107(45):19579-84.
Meguro M, Ito H, Takabayashi A, Tanaka R, Tanaka A (2011) Identification of the 7-Hydroxymethyl Chlorophyll a Reductase of the Chlorophyll Cycle in Arabidopsis. Plant Cell 23 (9):3442-3453.
Moon J, Parry G, Estelle M (2004). The ubiquitin-proteasome pathway and plant development. The Plant Cell, 16(12):3181-95.
Mora-Ramirez I, Weichert H, von Wiren N, Frohberg C, de Bodt S, Schmidt R C, Weber H (2021). The da1 mutation in wheat increases grain size under ambient and elevated CO2 but not grain yield due to trade-off between grain size and grain number. Plant-Environment Interactions, 2(2):61-73.
Nakagawa T, Suzuki T, Murata S, Nakamura S, Hino T, Maeo K, Tabata R, Kawai T, Tanaka K, Niwa Y, Watanabe Y (2007). Improved Gateway binary vectors: high-performance vectors for creation of fusion constructs in transgenic analysis of plants. Bioscience, biotechnology, and biochemistry, 71(8):2095-100.
Nguyen C D, Li J, Mou B, Gong H, Huo H (2021). A case study of using an efficient CRISPR/Cas9 system to develop variegated lettuce. Vegetable Research, 1(1):1-0.
Kerschbaum H H, Tasa B A, Schiirz M, Oberascher K, Bresgen N (2021). Trypan blue-Adapting a dye used for labelling dead cells to visualize pinocytosis in viable cells. Cell. Physiol. Biochem, 55:171-84.
Parry C, Blonquist Jr J M, Bugbee B (2014). In situ measurement of leaf chlorophyll concentration: analysis of the optical/absolute relationship. Plant, cell & environment, 37(11):2508-20.
Peng Y, Chen L, Lu Y, Wu Y, Dumenil J, Zhu Z, Bevan M W, Li Y (2015). The ubiquitin receptors DA1, DAR1, and DAR2 redundantly regulate endoreduplication by modulating the stability of TCP14/15 in Arabidopsis. The Plant Cell, 27(3):649-62.
Pfündel E, Neubohn B (1999). Assessing photosystem I and II distribution in leaves from C4 plants using confocal laser scanning microscopy. Plant, Cell & Environment, 22(12):1569-77.
Sato Y, Morita R, Katsuma S, Nishimura M, Tanaka A, Kusaba M (2009) Two short-chain dehydrogenase/reductases, NON-YELLOW COLORING 1 and NYC1-LIKE, are required for chlorophyll b and light-harvesting complex II degradation during senescence in rice. Plant J 57 (1):120-131.
Schindelin J, Rueden C T, Hiner M C, Eliceiri K W (2015). The ImageJ ecosystem: An open platform for biomedical image analysis. Molecular reproduction and development, 82(7-8):518-29.
Shim K C, Kim S H, Jeon Y A, Lee H S, Adeva C, Kang J W, Kim H J, Tai T H, Ahn S N (2020). A RING-type E3 ubiquitin ligase, OsGW2, controls chlorophyll content and dark-induced senescence in rice. International Journal of Molecular Sciences, 21(5):1704.
Shimoda Y, Ito H, Tanaka A (2016) Arabidopsis STAY-GREEN, Mendel's Green Cotyledon Gene, Encodes Magnesium-Dechelatase. Plant Cell 28 (9):2147-2160.
Song X J, Huang W, Shi M, Zhu M Z, Lin H X (2007). A QTL for rice grain width and weight encodes a previously unknown RING-type E3 ubiquitin ligase. Nature genetics, 39(5):623-30.
Thomas H, Ougham H (2014) The stay-green trait. Journal of Experimental Botany, 65(14):3889-900.
van Delden S H, SharathKumar M, Butturini M, Graamans L J A, Heuvelink E, Kacira M, Kaiser E, Klamer R S, Klerkx L, Kootstra G, Loeber A, Schouten R E, Stanghellini C, van Ieperen W, Verdonk J C, Vialet-Chabrand S, Woltering E J, van de Zedde R, Zhang Y, Marcelis L F M (2021) Current status and future challenges in implementing and upscaling vertical farming systems. Nat Food 2 (12):944-956.
Vanhaeren H, Nam Y J, De Milde L, Chae E, Storme V, Weigel D, Gonzalez N, Inze D (2017). Forever young: the role of ubiquitin receptor DA1 and E3 ligase BIG BROTHER in controlling leaf growth and development. Plant Physiology, 173(2):1269-82.
Wang P, Grimm B (2021) Connecting Chlorophyll Metabolism with Accumulation of the Photosynthetic Apparatus. Trends Plant Sci 26 (5):484-495. doi:10.1016/j.tplants.2020.12.005
Wang Z, Wang M, Yang C, Zhao L, Qin G, Peng L, Zheng Q, Nie W, Song C P, Shi H, Zhu J K (2021). SWO1 modulates cell wall integrity under salt stress by interacting with importin a in Arabidopsis. Stress Biology, 1:1-22.
Wickham H (2011). ggplot2. Wiley interdisciplinary reviews: computational statistics, 3(2):180-5.
Xia T, Li N, Dumenil J, Li J, Kamenski A, Bevan M W, Gao F, Li Y H (2013) The Ubiquitin Receptor DA1 Interacts with the E3 Ubiquitin Ligase DA2 to Regulate Seed and Organ Size in Arabidopsis. Plant Cell 25 (9):3347-3359.
Xie G, Li Z, Ran Q, Wang H, Zhang J (2018). Over-expression of mutated Zm DA1 or Zm DAR 1 gene improves maize kernel yield by enhancing starch synthesis. Plant Biotechnology Journal, 16(1):234-44.
Yang X, Han Y, Hao J, Qin X, Liu C, Fan S (2022). Exogenous spermidine enhances the photosynthesis and ultrastructure of lettuce seedlings under high-temperature stress. Scientia Horticulturae, 291:110570.
Yu H, Zhao J, Chen L, Wu T, Jiang B, Xu C, Cai Y, Dong J, Han T, Sun S, Yuan S (2023). CRISPR/Cas9-Mediated Targeted Mutagenesis of GmEOD1 Enhances Seed Size of Soybean. Agronomy, 13(9):2359.
Zhang J, Eswaran G, Alonso-Serra J, Kucukoglu M, Xiang J, Yang W, Elo A, Nieminen K, Damen T, Joung J G, Yun J Y (2019). Transcriptional regulatory framework for vascular cambium development in Arabidopsis roots. Nature plants, 5(10):1033-42.
Zhang Y, Lenhard M (2017). Exiting Already? Molecular Control of Cell-Proliferation Arrest in Leaves: Cutting Edge. Molecular Plant, 10(7):909-11.
Zhang J, Liu J, Yang C, Du S, Yang W (2016). Photosynthetic performance of soybean plants to water deficit under high and low light intensity. South African Journal of Botany, 105:279-87.
Zhang Y, Yin S, Tu Y, Mei H, Yang Y (2020). A novel microRNA, SlymiR208, promotes leaf senescence via regulating cytokinin biosynthesis in tomato. Physiologia Plantarum, 169(2):143-155
Zhou C E, Han L, Pislariu C, Nakashima J, Fu C X, Jiang Q Z, Quan L, Blancaflor E B, Tang Y H, Bouton J H, Udvardi M, Xia G M, Wang Z Y (2011) From Model to Crop: Functional Analysis of a STAY-GREEN Gene in the Model Legume Medicago truncatula and Effective Use of the Gene for Alfalfa Improvement. Plant Physiol 157 (3):1483-1496.

1. An oligonucleotide comprising a nucleic acid sequence encoding a guide RNA for targeting a promoter or exon region of LsDA1 gene from LOC111914107 on chromosome 6 or LOC111876247 on chromosome 1 in lettuce (Lactuca sativa), or a nucleic acid sequence with at least 95% sequence identity thereof.

2. The oligonucleotide of claim 1, wherein the guide RNA targets an exon region of LsDA1 gene from LOC111914107, optionally exon 7 or exon 8 on chromosome 6.

3. The oligonucleotide of claim 2, wherein the guide RNA comprises a spacer RNA sequence of SEQ ID NO:1 or SEQ ID NO:2, or an RNA sequence having at least 95% or 97% identity therewith.

4. An oligonucleotide comprising a nucleic acid sequence encoding a guide RNA for targeting a promoter or exon region of LsEOD1 gene from LOC111885075 on chromosome 1, LOC111877487 on chromosome 6, or LOC111881104 on chromosome 1 in lettuce (Lactuca sativa), or a nucleic acid sequence with at least 95% sequence identity thereof.

5. The oligonucleotide of claim 4, wherein the guide RNA targets an exon region of LsEOD1 gene from LOC111885075, optionally exon 1 or exon 2 on chromosome 1.

6. The oligonucleotide of claim 5, wherein the guide RNA comprises a spacer RNA sequence of SEQ ID NO:3 or SEQ ID NO:4, or an RNA sequence having at least 95% or 97% identity therewith.

7. A plasmid comprising a nucleic acid sequence encoding any of SEQ ID NOs:1-4 for cloning/replication or expression/transcription.

8. The plasmid of claim 7, wherein the plasmid further comprises a nucleic acid sequence encoding a Cas endonuclease, wherein the nucleic acid sequence is flanked at its 5′ end by an RNA polymerase II promoter sequence and at its 3′ end by a terminator sequence.

9. The plasmid of claim 7, wherein the plasmid further comprises nucleic acid sequences encoding a fluorescent protein and an antibiotic resistance marker, and wherein the nucleic acid sequences are separately or together flanked at its 5′ end by an RNA polymerase II promoter sequence and at its 3′ end by a terminator sequence.

10. The plasmid of claim 7,
wherein the plasmid is PHN-SpCas9-4×BsaI-GFP vector, comprising a nucleic acid sequence encoding a spacer RNA sequence of SEQ ID NO: 1 or 2 alone or with a nucleic acid sequence encoding a spacer RNA sequence of SEQ ID NO: 3 or 4,
wherein the nucleic acid sequence encoding any of SEQ ID NOs:1-4 is flanked at its 5′-end by a nucleic acid sequence encoding a tRNA and its 3′-end by a nucleic acid sequence encoding a scaffold RNA of SEQ ID NO:6, and
wherein the nucleic acid sequence encoding the first tRNA is preceded at its 5′-end by an RNA polymerase III promoter, and the nucleic acid sequence encoding the last scaffold RNA is followed at its 3′-end by a terminator sequence.

11. Agrobacterium comprising the plasmid of claim 7, the Agrobacterium optionally being Agrobacterium tumefaciens.

12. (canceled)

13. (canceled)

14. (canceled)

15. A method for generating lettuce having improved organ size and postharvest quality, the method comprising steps:
a. incubating cotyledon explants with Agrobacterium of claim 11;
b. culturing Agrobacterium-infected cotyledon explants in a callus-induction medium to produce calluses;
c. culturing the calluses of step b in a shoot-induction medium to produce shoots;
d. culturing the shoots of step c in a root induction medium; and
e. selecting transformed seedlings based on fluorescence in new roots and shoots.

16. Lettuce produced by the method of claim 15.
